(19) United States
(12) Patent Application Publication
(10) Pub. No.: US 2026/0241165 A1
(43) Pub. Date: Aug. 20, 2026

(54) ELECTRICAL STIMULATION SYSTEM FAULT DIAGNOSTICS

(71) Applicant: Cochlear Limited, Macquarie University, NSW (AU)

(72) Inventors: Rachel MACFARLANE, Dundas, NSW (AU); Samantha CONNOLLY, Middle Cove, NSW (AU)

(21) Appl. No.: 19/161,434

(22) PCT Filed: Mar. 4, 2024
(86) PCT No.: PCT/IB2024/052076

§ 371 Date: Sep. 2, 2025

Related U.S. Application Data
(60) Provisional application No. 63/489,304, filed on Mar. 9, 2023.

(51) Int. Cl. A61N 1/08; A61N 1/05; A61N 1/36; A61N 1/378

(52) U.S. Cl. CPC A61N 1/08; A61N 1/0541; A61N 1/36038; A61N 1/378; A61N 2001/083

(57) ABSTRACT

Presented herein are techniques for assessing proper functioning of an implantable component, such as an implantable medical device. In particular, an implant diagnostic system (integrity testing system) is configured to diagnose faults associated within an implantable electrical stimulation system. In certain embodiments, the implantable electrical stimulation system includes a plurality of stimulating electrodes configured to be implanted at a first location (first anatomical region) of a recipient. The integrity testing system includes one or more recording electrodes positioned at a second location (second anatomical region) of the recipient. The second location is a location that enables use of the one or more recording electrodes to detect phase reversals and other anomalous voltage patterns.

### BACKGROUND

### Field of the Invention

Presented herein are diagnostic techniques for implantable electrical stimulation systems.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: delivering, by an implantable electrical stimulation system, electrical stimulation signals to a first anatomical region of a recipient via a plurality of stimulating electrodes; recording one or more voltage responses induced in a body of the recipient resulting from the electrical stimulation signals, wherein the one or more voltage responses are captured using one or more recording electrodes disposed at a second anatomical region of the recipient that is located separately from the plurality of stimulating electrodes; and analyzing, by a processor, the one or more voltage responses to verify integrity of the implantable electrical stimulation system.

In another aspect, a system is provided. The system comprises an implantable electrical stimulation system configured to deliver electrical stimulation signals to a first anatomical region of a recipient via a plurality of electrodes that include at least one active electrode and at least one indifferent electrode; one or more recording electrodes configured to record one or more voltage responses induced in a body of the recipient resulting from the electrical stimulation signals, wherein the one or more recording electrodes are disposed at a second anatomical region of the recipient that is not between the at least one active electrode and the at least one indifferent electrode; and a processor configured to analyze the one or more voltage responses to verify integrity of the implantable electrical stimulation system.

In one aspect, a method is provided. The method comprises: delivering, by an implantable electrical stimulation system, electrical stimulation signals to a first anatomical region of a recipient via a plurality of electrodes of an electrode array; recording, by one or more recording electrodes disposed at a second anatomical region of the recipient that is located separately from the plurality of electrodes, one or more voltage responses induced in a body of the recipient resulting from the electrical stimulation signals; and sending the one or more voltage responses to a processor for determination of whether there is at least one of a fault associated with one or more of the plurality of electrodes or an abnormality associated with the electrode array or the first anatomical region of the recipient.

In another aspect, a system is provided. The system comprises: an implantable electrical stimulation system configured to deliver electrical stimulation signals to a first anatomical region of a recipient via a plurality of stimulating electrodes of an electrode array; one or more recording electrodes configured to record one or more voltage responses induced in a body of the recipient resulting from the electrical stimulation signals, wherein the one or more recording electrodes are disposed at a second anatomical region of the recipient located separately from the plurality of stimulating electrodes; and a processor configured to analyze the one or more voltage responses to determine whether there is at least one of a fault associated with one or more of the stimulating electrodes or an abnormality associated with the electrode array or the first anatomical region of the recipient.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 1E is a schematic diagram illustrating a computing device with which aspects of the techniques presented herein can be implemented;

FIG. 2A is a system diagram according to an example embodiment;

FIG. 2B is a system diagram according to another example embodiment, with a representation of the stimulus current generating a voltage response;

FIG. 3A is a common ground test scan plot for an implant, showing a normal response for a healthy cochlea and no evidence of malfunction;

FIG. 3B is a bipolar+1 test scan plot for an implant, showing a normal response for a healthy cochlea and no evidence of malfunction;

FIG. 3C is a monopolar 1 test scan plot for an implant, showing a normal response for a healthy cochlea and no evidence of malfunction;

FIG. 4A is a diagram showing normal current flow and the recorded voltage response when there is no evidence of malfunction in common ground mode;

FIG. 4B is a diagram showing abnormal current flow and the effect on the recorded voltage response when there is a short from electrode 6 to a stiffening ring in common ground mode;

FIG. 4C is a diagram showing normal current flow and the recorded voltage response when there is no evidence of malfunction when stimulating on electrode 4 in bipolar+1 mode;

FIG. 4D is a diagram showing abnormal current flow and the effect on the recorded voltage response when there is a short from electrode 6 to a stiffening ring when stimulating on electrode 4 in bipolar+1 mode;

FIG. 4E is a diagram showing normal current flow and the recorded voltage response when there is no evidence of malfunction when stimulating on electrode 6 in bipolar+1 mode;

FIG. 4F is a diagram showing abnormal current flow and the effect on the recorded voltage response when there is a short from electrode 6 to a stiffening ring when stimulating on electrode 6 in bipolar+1 mode;

FIG. 4G is a common ground test scan plot for an implant, showing an abnormal response when there is a short circuit from one or more stimulating electrodes (electrodes 9, 19, and 22 in this example) to a stiffening ring in common ground mode;

FIG. 4H is a bipolar+1 test scan plot for an implant, showing an abnormal response when there is a short circuit from one or more stimulating electrodes (electrodes 9, 19, and 22 in this example) to a stiffening ring in bipolar+1 mode;

FIG. 5A is a diagram showing current flow and how a short between electrodes in common ground mode causes current to flow through tissue (rather than the short) when stimulating on a non-shorted electrode (electrode 3 in this example);

FIG. 5B is a diagram showing current flow and how a short between electrodes in common ground mode causes current to flow through the short (rather than tissue) when stimulating on the shorted electrode (electrode 4 in this example);

FIG. 5C is a diagram showing normal current flow and the recorded voltage response when there is no evidence of malfunction when stimulating on electrode 2 in bipolar+1 mode;

FIG. 5D is a diagram showing abnormal current flow and the effect on the recorded voltage response when there is a short from electrode 4 to electrode 8 when stimulating on electrode 2 in bipolar+1 mode;

FIG. 5E is a diagram showing normal current flow and the recorded voltage response when there is no evidence of malfunction when stimulating on electrode 4 in bipolar+1 mode;

FIG. 5F is a diagram showing abnormal current flow and the effect on the recorded voltage response when there is a short from electrode 4 to electrode 8 when stimulating on electrode 4 in bipolar+1 mode;

FIG. 5G is a diagram showing normal current flow and the recorded voltage response when there is no evidence of malfunction when stimulating on electrode 6 in bipolar+1 mode;

FIG. 5H is a diagram showing abnormal current flow and the effect on the recorded voltage response when there is a short from electrode 4 to electrode 8 when stimulating on electrode 6 in bipolar+1 mode;

FIG. 5I is a diagram showing normal current flow and the recorded voltage response when there is no evidence of malfunction when stimulating on electrode 8 in bipolar+1 mode;

FIG. 5J is a diagram showing abnormal current flow and the effect on the recorded voltage response when there is a short from electrode 4 to electrode 8 when stimulating on electrode 8 in bipolar+1 mode;

FIG. 5K is a common ground test scan plot for an implant, showing an abnormal response when there is a short circuit between two electrodes (electrode 8 to electrode 21 in this example) in common ground mode;

FIG. 5L is a bipolar+1 test scan plot for an implant, showing an abnormal response when there is a short circuit between two electrodes (electrode 8 to electrode 21 in this example) in bipolar+1 mode;

FIG. 6A is a common ground test scan plot for an implant, showing an abnormal response (which appears normal or near normal) when the electrode array is completely outside the cochlea;

FIGS. 6B and 6C are monopolar 1 and monopolar 2 test scan plots for an implant, showing an abnormal response when the electrode array is completely outside the cochlea;

FIG. 6D is a common ground test scan plot for an implant, showing an abnormal response when the electrode array is partially extruded from the cochlea;

FIG. 6E is a bipolar+1 test scan plot for an implant, showing an abnormal response when the electrode array is partially extruded from the cochlea;

FIG. 6F is a common ground test scan plot for an implant, showing an abnormal response in the case of cochlear otospongiosis (spongy cochlea bone) in common ground mode;

FIG. 6G is a bipolar+1 test scan plot for an implant, showing an abnormal response in the case of cochlear otospongiosis (spongy cochlea bone) in bipolar+1 mode;

FIG. 6H shows a series of four common ground test scan plots taken using an electrode array inserted into a model of a cochlea immersed in saline to simulate different responses ranging from a healthy insulating cochlea (solid walls) in the top plot to a spongy conductive cochlea (porous walls) in the bottom plot;

FIG. 6I shows a series of panels illustrating current flow and the effect on the recorded voltage response in phase two of the stimulation pulse for a normal healthy cochlea (left hand side) and a spongy cochlea (right hand side) when stimulating in three different positions along the electrode array in bipolar+1 mode;

FIG. 6J is a common ground test scan plot for an implant, showing a suspect response in the case of a suspected spongy cochlea in common ground mode;

FIG. 6K is a monopolar 1 test scan plot for an implant, showing a suspect response in the case of a suspected spongy cochlea in monopolar 1 mode;

FIG. 7 is a schematic diagram illustrating a vestibular stimulator system with which aspects of the techniques presented herein can be implemented;

FIG. 8 is a schematic diagram illustrating an implantable stimulator system and a wearable device with which aspects of the techniques presented herein can be implemented;

FIG. 9A is a common ground test scan plot for an implant, showing a normal voltage response in the case of a healthy cochlea with no evidence of malfunction in common ground mode;

FIG. 9B is a bipolar+1 test scan plot for an implant, showing a normal voltage response in the case of a healthy cochlea with no evidence of malfunction in bipolar+1 mode;

FIG. 9C is a common ground test scan plot for an implant, showing an abnormal voltage response and illustrating how to interpret the abnormal voltage response in common ground mode;

FIG. 9D is a bipolar+1 test scan plot for an implant, showing an abnormal voltage response and illustrating how to interpret the abnormal voltage response in bipolar+1 mode;

FIG. 10 is a flowchart illustrating a method for verifying integrity of an implantable electrical stimulation system, according to an example embodiment; and

FIG. 11 is a flowchart illustrating a method for determining whether there is at least one of a fault associated with one or more stimulating electrodes or an abnormality associated with the electrode array or the first anatomical region of the recipient, according to an example embodiment.

### DETAILED DESCRIPTION

### Overview

Presented herein are techniques for assessing proper functioning of an implantable component (implantable medical device). In particular, an implant diagnostic system, also sometimes referred to herein as an integrity testing system, is configured to diagnose faults associated within an implantable electrical stimulation system. In certain embodiments, the electrical stimulation system includes stimulating electrodes configured to be implanted at a first location (e.g., the head) of a recipient, also referred to as a first anatomical region herein. The integrity testing system includes one or more recording electrodes positioned at a second location of the recipient, also referred to as a second anatomical region herein. The second location is a location that enables use of the one or more recording electrodes to detect phase reversals and other anomalous voltage patterns. That is, by ensuring that there is sufficient distance between the electrical stimulation system and the recording electrodes, it is possible to detect phase reversals, which can indicate a short circuit, and/or other anomalous voltage patterns.

The integrity testing systems and methods described herein at least partially integrate the testing capability for detecting anomalous voltage patterns (e.g., phase reversals) into an implantable device/component (implantable medical device), such as a cochlear implant. In operation, the integrity testing system is configured to use in situ electrical measurements (e.g., voltage and/or current measurements) to identify whether there are any malfunctions or other abnormalities, including but not limited to detecting phase reversals that indicate the presence of a short circuit fault in the implant. Using the techniques described in detail below, a cochlear implant or other implantable device cooperate with a measurement component located elsewhere in or on the body of the recipient (e.g., recording electrodes implanted in the chest of the recipient) to detect, for example, phase reversals. The integrity testing system integrated with the implantable device can, for example, include the ability to self-diagnose various stimulation faults, classify types of faults, cross-reference with other tests results or supplemental data to confirm a fault hypothesis or distinguish between different types of faults and other abnormalities, etc.

Merely for ease of description, the techniques presented herein are primarily described with reference to a specific implantable medical device system, namely a cochlear implant system. However, it is to be appreciated that the techniques presented herein can also be partially or fully implemented by other types of implantable medical devices. For example, the techniques presented herein can be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein can also be implemented by dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

### Example Implant System

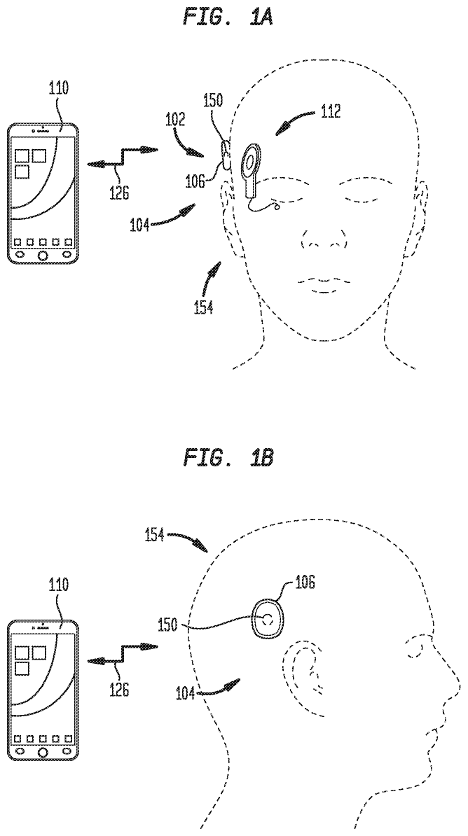

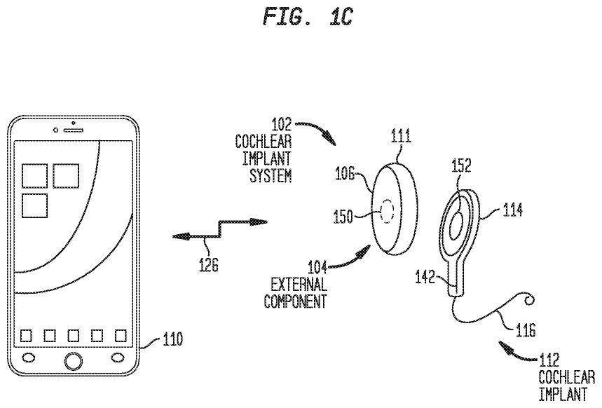

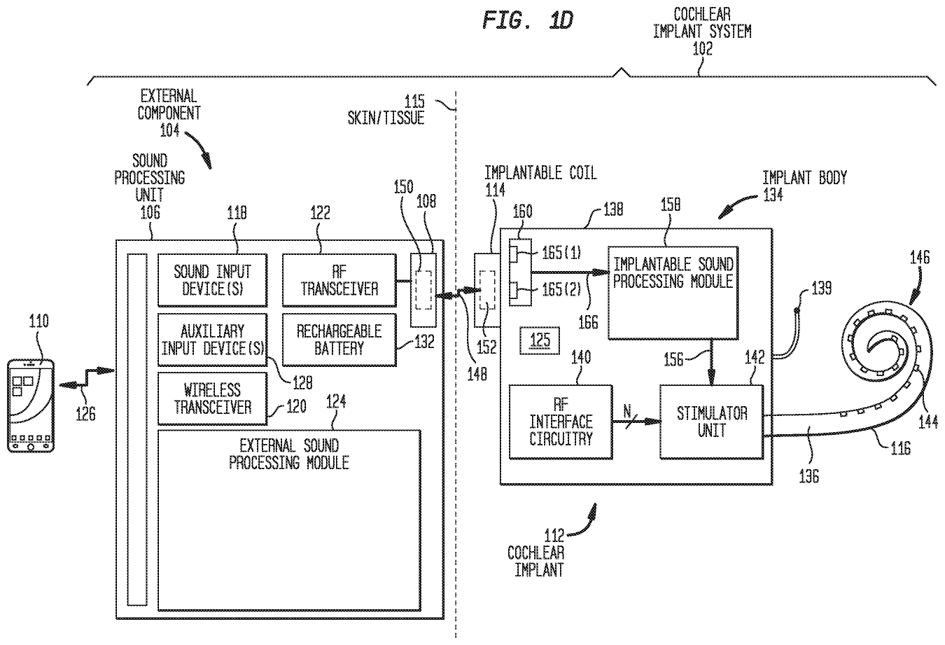

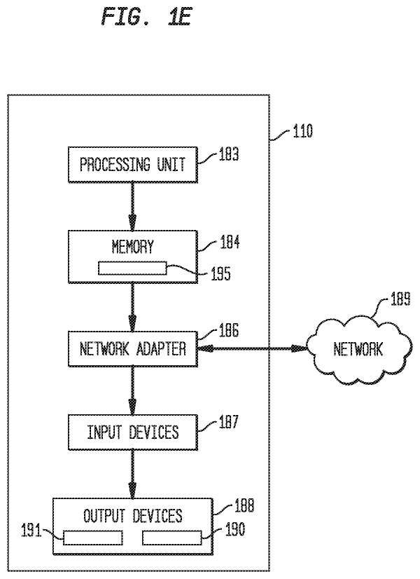

FIGS. 1A-1D illustrates an example cochlear implant system 102 with which aspects of the techniques presented herein can be implemented. The cochlear implant system 102 comprises an external component 104 that is configured to be directly or indirectly attached to the body of the recipient, and an internal/implantable component 112 that is configured to be implanted in or worn on the head of the recipient. In the examples of FIGS. 1A-1D, the implantable component 112 is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

In the examples of FIGS. 1A-1D, the external component 104 comprises a sound processing unit 106, an external coil 108, and generally, a magnet fixed relative to the external coil 108. The cochlear implant 112 includes an implantable coil 114, an implant body 134, and an elongate stimulating assembly 116 configured to be implanted in the recipient's cochlea. In one example, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the recipient's head 154 (e.g., includes an integrated external magnet 150 configured to be magnetically coupled to an internal/implantable magnet 152 in the implantable component 112). The OTE sound processing unit 106 also includes an integrated external (headpiece) coil 108 (the external coil 108) that is configured to be inductively coupled to the implantable coil 114.

It is to be appreciated that the OTE sound processing unit 106 is merely illustrative of the external devices that could operate with implantable component 112. For example, in alternative examples, the external component 104 can comprise a behind-the-ear (BTE) sound processing unit configured to be attached to, and worn adjacent to, the recipient's ear. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

Although the cochlear implant system 102 includes the sound processing unit 106 and the cochlear implant 112, as described below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110, which is shown in greater detail in FIG. 1E, is a computing device, such as a personal computer (e.g., laptop, desktop, tablet), a mobile phone (e.g., smartphone), remote control unit, etc. The external device 110 and the cochlear implant system 102 (e.g., sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc.

Returning to the example of FIGS. 1A-1D, the sound processing unit 106 of the external component 104 also comprises one or more input devices configured to capture and/or receive input signals (e.g., sound or data signals) at the sound processing unit 106. The one or more input devices include, for example, one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a short-range wireless transmitter/receiver (wireless transceiver) 120 (e.g., for communication with the external device 110), each located in, on or near the sound processing unit 106. However, it is to be appreciated that one or more input devices can include additional types of input devices and/or less input devices (e.g., the short-range wireless transceiver 120 and/or one or more auxiliary input devices 128 could be omitted).

The sound processing unit 106 also comprises the external coil 108, a charging coil 130, a closely-coupled radio frequency transmitter/receiver (RF transceiver) 122, at least one rechargeable battery 132, and an external sound processing module 124. The external sound processing module 124 can be configured to perform a number of operations and can be formed by one or more processors (e.g., one or more Digital Signal Processors (DSPs), one or more uC cores, etc.), firmware, software, etc. arranged to perform operations described herein. That is, the external sound processing module 124 can be implemented as firmware elements, partially or fully implemented with digital logic gates in one or more application-specific integrated circuits (ASICs), partially or fully in software, etc.

In the example of FIGS. 1A-1D, the implantable component 112 comprises an implant body (main module) 134, a lead region 136, and the intra-cochlear stimulating assembly 116, all configured to be implanted under the skin (tissue) 115 of the recipient. The implant body 134 generally comprises a hermetically-sealed housing 138 that includes, in certain examples, at least one power source 125 (e.g., one or more batteries, one or more capacitors, etc.) 125, in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact array (electrode array) 146 for delivery of electrical stimulation (current) to the recipient's cochlea. Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 150 is fixed relative to the external coil 108 and the internal/implantable magnet 152 is fixed relative to the implantable coil 114. The external magnet 150 and the internal/implantable magnet 152 fixed relative to the external coil 108 and the internal/implantable coil 114, respectively, facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to process the received input audio signals (received at one or more of the input devices, such as sound input devices 118 and/or auxiliary input devices 128), and convert the received input audio signals into output control signals for use in stimulating a first ear of a recipient (i.e., the external sound processing module 124 is configured to perform sound processing on input signals received at the sound processing unit 106). Stated differently, the one or more processors (e.g., processing element(s) implementing firmware, software, etc.) in the external sound processing module 124 are configured to execute sound processing logic in memory to convert the received input audio signals into output control signals (stimulation signals) that represent electrical stimulation for delivery to the recipient.

As noted, FIG. 1D illustrates an embodiment in which the external sound processing module 124 in the sound processing unit 106 generates the output control signals. In an alternative embodiment, the sound processing unit 106 can send less processed information (e.g., audio data) to the implantable component 112 and the sound processing operations (e.g., conversion of input sounds to output control signals 156) can be performed by a processor within the implantable component 112.

In FIG. 1D, according to an example embodiment, output control signals (stimulation signals) are provided to the RF transceiver 122, which transcutaneously transfers the output control signals (e.g., in an encoded manner) to the implantable component 112 via external coil 108 and implantable coil 114. That is, the output control signals (stimulation signals) are received at the RF interface circuitry 140 via implantable coil 114 and provided to the stimulator unit 142. The stimulator unit 142 is configured to utilize the output control signals to generate electrical stimulation signals (e.g., current signals) for delivery to the recipient's cochlea via one or more of the stimulating contacts (electrodes) 144. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the input audio signals (the received sound signals).

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, an example embodiment of the cochlear implant 112 can include a plurality of implantable sound sensors 165(1), 165(2) that collectively form a sensor array 160, and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device. In an alternative implementation of the external hearing mode, the cochlear implant 112 could use signals captured by the sound input devices 118 and the implantable sound sensors 165(1), 165(2) of sensor array 160 in generating stimulation signals for delivery to the recipient.

In the invisible hearing mode, the implantable sound sensors 165(1), 165(2) of the sensor array 160 are configured to detect/capture input sound signals 166 (e.g., acoustic sound signals, vibrations, etc.), which are provided to the implantable sound processing module 158. The implantable sound processing module 158 is configured to convert received input sound signals 166 (received at one or more of the implantable sound sensors 165(1), 165(2)) into output control signals 156 for use in stimulating the first ear of a recipient (i.e., the implantable sound processing module 158 is configured to perform sound processing operations). Stated differently, the one or more processors (e.g., processing element(s) implementing firmware, software, etc.) in implantable sound processing module 158 are configured to execute sound processing logic in memory to convert the received input sound signals 166 into output control signals 156 that are provided to the stimulator unit 142. The stimulator unit 142 is configured to utilize the output control signals 156 to generate electrical stimulation signals (e.g., current signals) for delivery to the recipient's cochlea, thereby bypassing the absent or defective hair cells that normally transduce acoustic vibrations into neural activity.

FIG. 1E is a block diagram illustrating one example arrangement for an external computing device 110 configured to perform one or more operations in accordance with certain embodiments presented herein. As shown in FIG. 1E, in its most basic configuration, the external computing device 110 includes at least one processing unit 183 and a memory 184. The processing unit 183 includes one or more hardware or software processors (e.g., Central Processing Units) that can obtain and execute instructions. The processing unit 183 can communicate with and control the performance of other components of the external computing device 110. The memory 184 is one or more software or hardware-based computer-readable storage media operable to store information accessible by the processing unit 183. The memory 184 can store, among other things, instructions executable by the processing unit 183 to implement applications or cause performance of operations described herein, as well as other data. The memory 184 can be volatile memory (e.g., RAM), non-volatile memory (e.g., ROM), or combinations thereof. The memory 184 can include transitory memory or non-transitory memory. The memory 184 can also include one or more removable or non-removable storage devices. In examples, the memory 184 can include random access memory (RAM), read only memory (ROM), EEPROM (Electronically-Erasable Programmable Read-Only Memory), flash memory, optical disc storage, magnetic storage, solid state storage, or any other memory media usable to store information for later access. By way of example, and not limitation, the memory 184 can include wired media such as a wired network or direct-wired connection, and wireless media such as acoustic, RF, infrared and other wireless media or combinations thereof. In certain embodiments, the memory 184 comprises integrity testing logic 195 that, when executed, enables the processing unit 183 to perform aspects of the implant diagnostic techniques presented.

In the illustrated example of FIG. 1E, the external computing device 110 further includes a network adapter 186, one or more input devices 187, and one or more output devices 188. The external computing device 110 can include other components, such as a system bus, component interfaces, a graphics system, a power source (e.g., a battery), among other components. The network adapter 186 is a component of the external computing device 110 that provides network access (e.g., access to at least one network 189). The network adapter 186 can provide wired or wireless network access and can support one or more of a variety of communication technologies and protocols, such as ETHERNET, cellular, BLUETOOTH, near-field communication, and RF (Radiofrequency), among others. The network adapter 186 can include one or more antennas and associated components configured for wireless communication according to one or more wireless communication technologies and protocols. The one or more input devices 187 are devices over which the external computing device 110 receives input from a user. The one or more input devices 187 can include physically-actuatable user-interface elements (e.g., buttons, switches, or dials), a keypad, keyboard, mouse, touchscreen, and voice input devices, among other input devices that can accept user input. The one or more output devices 188 are devices by which the computing device 110 is able to provide output to a user. The output devices 188 can include, for example, a display 190 (e.g., a liquid crystal display (LCD)) and one or more speakers 191, among other output devices for presentation of visual or audible information to the recipient, a clinician, an audiologist, or other user. For example, various integrity test scan plots showing recorded voltage responses can be presented on the display 190.

It is to be appreciated that the arrangement for the external computing device 110 shown in FIG. 1E is merely illustrative and that aspects of the techniques presented herein can be implemented at a number of different types of systems/devices including any combination of hardware, software, and/or firmware configured to perform the functions described herein. For example, the external computing device 110 can be a personal computer (e.g., a desktop or laptop computer), a hand-held device (e.g., a tablet computer), a mobile device (e.g., a smartphone), a surgical system, and/or any other electronic device having the capabilities to perform the associated operations described elsewhere herein.

### Example Integrity Testing Systems

As noted, an implant diagnostic system (integrity testing system) described herein can be integrated with implantable devices that include an electrical stimulation system comprising a plurality of stimulating electrodes configured to be implanted in/at a first location of a body (e.g., a first anatomical region, such as the head) of a recipient, and one or more recording electrodes (and potentially other parts) configured to be positioned at a second location of the body (e.g., a second anatomical region, such as the chest or other body part) of the recipient. As described elsewhere herein, the one or more recording electrodes are positioned at a sufficient distance from the stimulating electrodes to permit the recording electrodes to detect anomalous voltage patterns, such as phase reversals. By ensuring that there is at least a minimum threshold separation distance between the electrical stimulation system (e.g., the stimulating electrodes) and the recording electrodes (e.g., implanted in, attached to, or worn on the body of the recipient), it is possible to detect anomalous voltage patterns, such as phase reversals, which indicate problems with the electrical stimulation system (e.g., a short circuit, etc.). In addition, there can also be a maximum threshold separation distance between the stimulating electrodes and the recording electrodes for detecting and measuring the anomalous voltage patterns.

It should be appreciated that the recording electrodes as described herein do not refer to the indifferent electrode (as distinguished from the active stimulating electrode), i.e., the extracochlear electrodes (ECEs) when in monopolar mode or the intracochlear electrodes (ICEs) when in bipolar mode or common ground mode. The recording electrode(s) is/are implemented using one or more other electrode(s) besides the ECEs or ICEs of the cochlear implant.

In certain embodiments presented herein, to test integrity of the implantable device, a pre-defined stimulus signal is sent via the RF link to generate electrical stimulation at the cochlear implant. Thereafter, measurement data (e.g., voltage measurements) is captured and used to detect the presence of faults or problems with the electrical stimulation system. For example, the measurement data can be transferred to a processor (e.g., a processor in the non-implanted external component of the device, or a processor in an external computing device), which alerts the recipient or other user when faults in the implant are detected.

In some examples, the recording electrode is located in or on (e.g., implanted in, attached to, or worn on) the chest of the recipient. In some other examples, the recording electrode could be located elsewhere in or on the body of the recipient, where this location can be any body part that is far enough away from the site of electrical stimulation by the implantable component to record an anomalous voltage pattern, such as a phase reversal in a voltage response, yet still close enough to be able to record the voltage response.

In summary, aspects presented herein relate to an integrity testing system for cochlear implants or other implantable devices, and corresponding method for performing self-testing/self-diagnostic operations of the device. The techniques presented herein can use of the direction of current flow, the amplitude of voltage responses, and/or the presence of phase reversals to evaluate the function/operation/integrity of the cochlear implant, such as by identifying the existence of short circuit faults in its stimulating electrodes, an extruded array, a spongy cochlea, or other abnormalities as described in detail below.

Referring again to FIGS. 1A-1E, the testing can utilize a diagnostic data analysis process on board the implant (e.g., an internal/implanted processor) in some examples. In some other examples, the testing can utilize a diagnostic data analysis process on an external component connected with the implanted component, or on an external computing device (e.g., a computer, a tablet, a smartphone, etc.) in communication with the implantable device.

In some examples, initial testing can be a manual implementation operated by a trained professional, and this initial manual testing can be later followed by routine automatic testing which does not require availability of trained operators. In some other examples, testing could be automated to occur without requiring any trained operator involvement.

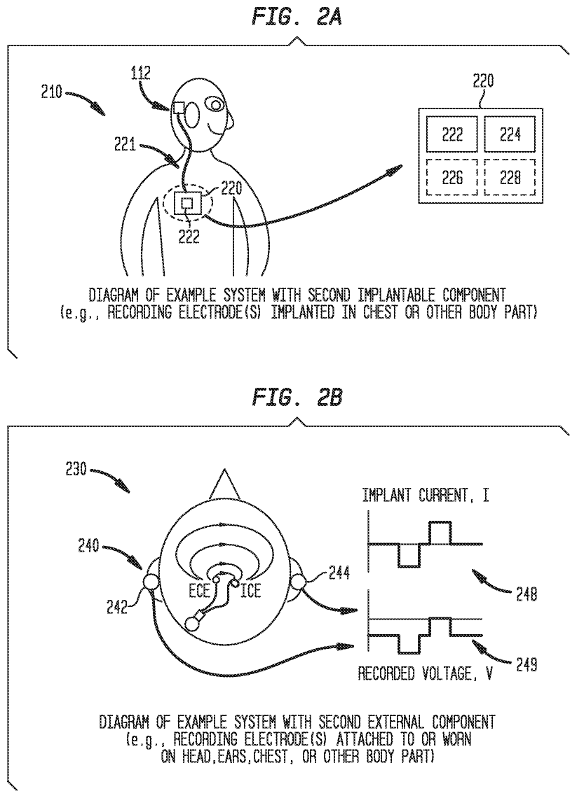

Now referring to FIGS. 2A and 2B, several different example implementations for the integrity testing system and methods are described.

FIG. 2A is a system diagram illustrating an integrity testing system 210 according to an example embodiment. The integrity testing system 210 includes a first implantable component 112 and a second implantable component 220. The second implantable component 220 can be separate from the first implantable component 112, or electrically connected to the first implantable component 112 via a subcutaneous lead wire 221, for example. The second implantable component 220 includes one or more recording electrodes 222 and a battery 224, at a minimum. In some example embodiments, the second implantable component 220 can additionally include an amplifier (not shown), a processor 226, and a communications interface 228 (e.g., to an external processor). If there is no subcutaneous lead wire connecting the second implantable component 220 to the first implantable component 112, then the second implantable component 220 would also include a memory and firmware to store measurements and communicate the information out to compatible devices.

According to an aspect, the integrity testing system 210 and corresponding methods can involve placing a part of the implant system 102 in another body part of the recipient along with the implantable component 112 of the implant system 102 that is placed in the head. More specifically, in addition to the stimulating electrodes (e.g., intracochlear electrodes (ICEs) or extracochlear electrodes (ECEs)), one or more other implantable electrodes of the integrity testing system can be separately implanted in a body part other than the recipient's head, such as the recipient's chest or other body part, to function as the one or more recording electrodes 222.

According to another aspect, a distance/location condition is that the recording electrode(s) 222 should not sit between the active electrode and the indifferent electrode of the implantable component (e.g., the extracochlear electrodes (ECEs) in monopolar mode, or the intracochlear electrodes (ICEs) in bipolar mode or common ground mode). This is due to the system's reliance on detecting the current spread outwards from the flow between the active electrode and the indifferent electrode of the implant. In some example embodiments, a minimum/maximum separation distance between the stimulating electrodes (ICEs or ECEs) and the recording electrode(s) 222 can be derived from two elements: (1) the noise in the electrical system setting a minimum limit of voltage detection, and/or (2) the current level of the stimulation being detected. For example, a higher current level would mean the recording electrode could be located further away from the stimulating electrodes (whereas the recording electrode could be located closer to the stimulating electrodes in the case of a lower current level). In other words, a separation distance threshold (or target range) can be set or adjusted as a function of the current level and any system noise. The key issue is that recording electrode placement cannot be in the current path. A minimum distance of at least a few centimetres away (e.g., ~5 cm distance in one example implementation) is better to receive sufficient spread of current. If the distance is too large, the signal will be too weak to use effectively, so defining an upper limit on the distance may require testing first to determine if an implant location somewhere lower in the recipient's body would be sufficient.

In example embodiments using one or more recording electrodes 222 that are implanted under the skin surface of the recipient (e.g., in the chest or other body part at a sufficient distance from the stimulating electrodes in the head), electrode pads on the surface of the skin (skin patch electrodes) are not needed. The concept of providing a chest-implanted electrode as the recording electrode also allows for a new direction of current spread to be detected (as opposed to side-to-side of the head as in FIG. 2B described below), which can provide enhanced diagnostic value.

In some other example embodiments in which the recording electrodes are integrated in an external device (e.g., a skin patch or a wearable device), the system and methods can utilize an electrode pad or skin patch to adhere the recording electrodes to the skin surface of the recipient and ensure that sufficient contact therebetween is maintained throughout the testing operations, as described below with reference to FIG. 2B.

FIG. 2B is a system diagram illustrating an integrity testing system 230 according to an example embodiment. The integrity testing system 230 includes an implantable component 112 and an external component 240 including a pair of recording electrodes 242 and 244. FIG. 2B shows a view from above the recipient's head with the ears on the sides and the nose to the front. A pair of recording electrodes 242 and 244 (shown as large black dots) are attached to the ears. Inside the head, a cochlear implant 112 is shown with an extracochlear electrode (ECE) 1 (e.g., a ball electrode) to the left and an intracochlear electrode (ICE) on the right. Elliptical lines flowing between the two electrodes represent current flowing from the extracochlear electrode (ECE) 1 to the intracochlear electrode (ICE). Thicker lines represent larger currents (and for illustrative purposes are shown anterior to the electrodes). Although some of the current flows directly between the two electrodes, some of the current spreads away from the site of stimulation to the recording electrodes 242 and 244, where the current is detected, amplified, and recorded. To the right of the head, a sketch of the current flow 248 and the coincident voltage response 249 that is recorded as a result of that current flow.

As stated, the recording electrodes 242 and 244 can be one or more body-worn electrodes separately attached to or worn on the other body part of the recipient (e.g., the chest, the ears, the neck, the forehead, etc.). For example, the external component 240 (e.g., the recording electrodes 242 and 244) can be integrated in an electrode pad or skin patch having an adhesive surface for securely attaching the external component 240 to the skin of the recipient (e.g., so that current can be detected at the skin surface).

In the case of a cochlear implant, all of the components for implementing the techniques described herein could in theory be implanted in the recipient's head (as opposed to being attached to the head or ears, or worn on the head), so long as the recording electrode satisfies the aforementioned condition of not being located too close to the stimulating electrodes (i.e., not positioned between the active electrode and the indifferent electrode) of the implant. When detecting phase reversals, the location of the recording electrode relative to the stimulating electrodes (the active and indifferent electrodes) can in some instances have greater importance than the specific location (body part) itself.

Although FIG. 2B shows recording electrodes 242 and 244 attached to the sides of the head (e.g., the ears), different locations on the head can be used for attachment in other examples, or the recording electrodes could be implanted in the head (in accordance with the threshold separation distance constraint) in other examples.

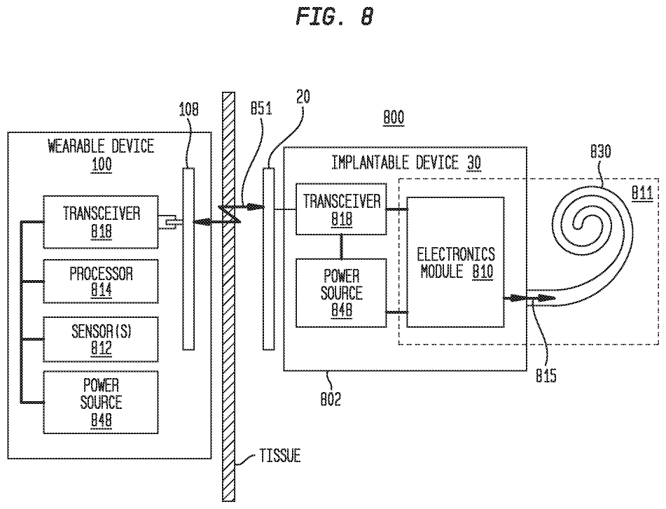

In a variation of the example embodiment of FIG. 2B, the integrity testing system 230 may be implemented with a wearable component that includes one or more recording electrodes. In some example embodiments, the recording electrode(s) can be incorporated into an externally worn wearable device, which communicates with the implantable device so that the wearable device and/or the implant can detect a phase reversal. Examples of various externally attached or body-worn devices that can incorporate the external component(s) can include smart watches, fitness trackers, behind-the-ear devices (BTEs, including contra-lateral BTEs), etc. Various details of an externally worn wearable device 100 are described below with reference to FIG. 8.

It is also noted, however, that any external components of the integrity testing system (e.g., recording electrodes integrated in a skin patch or an externally worn device) should provide good contact with and a sufficiently robust connection to the skin surface during testing operations. For example, a typical smartwatch or fitness tracker which are worn somewhat loosely, can utilize some further modification or adaptation to provide a sufficiently robust connection. Similarly, a standard BTE device may not provide sufficient skin contact absent some modification/adaptation. Therefore, a patch electrode and a cleaning process (whereby the recipient's skin is cleaned with sanitizing wipes and light exfoliation before the patch electrode is placed on the skin) can be used to provide a sufficiently robust connection to the skin surface, in example embodiments where the recording/measurement electrodes are externally attached or body-worn (instead of being implanted under the skin surface).

As described above with reference to FIGS. 1A-1E and FIGS. 2A and 2B, some example embodiments provide an implant diagnostic system that is largely integrated with (but not necessarily completely integrated into) the cochlear implant or other implantable device (e.g., electrically connected with the internal/implantable component thereof). Components that are integrated into or with the cochlear implant can be augmented by one or more other external component(s) that can be attached to the skin or worn externally on the body of the recipient. Some other example embodiments provide an external standalone implant diagnostic system (e.g., an external computing device programmed to analyze and interpret the results of various integrity tests) to detect phase reversals, and therefore, to detect short circuit faults in implants as well as various other abnormalities as described herein. Generally, the system and methods described herein allow for real time constant monitoring of short circuits and other issues in an implantable hearing device, such as a cochlear implant, and are designed to ensure that implant failures or performance decrements can be detected immediately upon the occurrence thereof.

Having the measurement and stimulus process integrated with the implantable device removes the inconvenience of transporting specialist hardware for diagnosing implant failures and removes hardware complexity involved in synchronizing test steps. The systems and methods described herein also remove any need to set up an appointment where specialised equipment and expertise can be accessed. The systems and methods described herein remove any need for a trigger signal between the stimulating devices (e.g., the stimulator and the stimulating electrodes) and the recording devices (e.g., the recording electrodes), since both of these operations occur in the same device/system.

To further understand the structural/hardware and functional/software aspects of the system for testing integrity of a cochlear implant and methods for interpretation of electrical measurements from the integrity test system, various background theoretical information and relevant technical implant characteristics are described in detail below. Additionally, signature patterns of different classifications (ranging from “normal” conditions to various commonly seen “abnormal” conditions) are also described, as well as various failure modes or faults that can occur and how these can appear in different integrity test scan results.

In accordance with embodiments presented herein, to establish whether an implantable electrical stimulation system (e.g., of a cochlear implant), is functioning as expected, recording electrodes can be used to record voltages induced in the body of the implant recipient resulting from current pulses delivered by the electrical stimulation system (e.g., the cochlear implant). The recorded voltages, in turn, can be analyzed to identify possible faults in the implant. At a basic level, the integrity testing system (e.g., any of integrity testing systems 210 or 230 of FIG. 2A or 2B) includes an integrity test unit (hardware device(s) for data acquisition), integrity test software (to perform the tests), and analysis software (to view and interpret the integrity test results). The integrity test system measures voltages between the recording electrodes and the cochlear implant, wherein current generated by the cochlear implant flows in the body tissue/compounds/fluid between the stimulating electrodes.

If the electrical stimulation system of an implant is functioning correctly, then an integrity test should reveal a series of biphasic square wave pulses corresponding to the current pulses delivered by the implant. An aspect of interpreting an integrity test involves looking at the relative magnitude (size) and phase (negative or positive compared to the current) of these pulses as one or more parameters is varied. Five factors that influence the magnitude and phase of the response associated with a cochlear implant are described below, where the effects of which can be used to explain the results seen in integrity tests. These five factors include:

(1) Effect of stimulus current on response. The amplitude of the recorded voltage response is proportional to the stimulus current being produced by the implant. The distance between the stimulating electrode and the recording electrode will affect the magnitude of the charge recorded in a nonlinear way. For example, if the stimulus current is doubled then the recorded voltage should show a corresponding increase, or if the current is halved then the voltage should show a corresponding decrease. If the phase of the current is reversed, as happens between phase one and phase two of the biphasic stimulus pulse, then the phase of the recorded voltage should also be reversed.

(2) Effect of distance between stimulating electrodes on response. The farther apart the stimulating electrodes, the larger the response. The closer together the stimulating electrodes, the smaller the response.

(3) Effect of depth of stimulating electrode(s) in the cochlea on response. For bipolar stimulation modes, how far the electrode pair is from the cochleostomy is another factor in determining the size of the recorded response. Generally, the most basal electrode (closest to the cochleostomy) out of the bipolar pair of electrodes dominates. In a healthy cochlea, the bone surrounding the scalae forms an insulating tube which allows no (or very little) of the current to escape from it. Therefore, the main source of current responsible for registering as a response in stimulation modes where current flows only inside the cochlea (bipolar and common ground modes) is from current escaping from the cochleostomy. If the electrode pair is deeper inside the cochlea (more apical), less current will escape from the cochleostomy and the response will be smaller. Conversely, if the electrode pair is shallower in the cochlea (more basal), more current will escape from the cochleostomy and the response will be larger. In the particular case of a “spongy” cochlea (a condition associated with cochlear otosclerosis, as described further below), the cochlear bone can be less dense (or porous) and consequently, more conductive, such that current is not completely confined within the walls of the cochlea and a significant portion can escape and influence the recorded response.

(4) Effect of direction of current flow on the response. The responses produced by currents flowing in the cochlea are dependent on the direction of current flow along the scalae (i.e., whether the current flow is apical to basal, or basal to apical). In the case of a “spongy” (more conductive) cochlea, the geometry of the cochlea and relative position of any extracochlear electrodes becomes important. In the case of monopolar stimulation, for example, current is passed between an extracochlear electrode (ECE) and an intracochlear electrode (ICE). In a healthy cochlea, almost all the current is formed to flow through the cochleostomy to get from the ECE to the ICE, so the position of the intracochlear electrode has very little impact on the recorded response. In a spongy cochlea, however, current can flow directly from the extracochlear electrode to the intracochlear electrode. Thus, the direction of the current flow is different for different electrodes, which this leads to a different response size and/or phase. Given the orientation of the recording electrode(s), the amplitude of the recorded response in the case of a healthy cochlea is relatively smaller, and the amplitude of the recorded response in the case of a spongy cochlea is relatively larger. In the case of a spongy cochlea, choosing a different intracochlear electrode with a different position in the cochlea will change the orientation of the current paths and lead to a different response amplitude. Likewise, in bipolar mode as the electrode pair is moved, the orientation of the current paths will also change. In a very spongy cochlea, the direction of the current path can change by 180 degrees, leading to a complete phase reversal in the recorded response.

(5) Effect of superposition of responses from two or more current paths. For many stimulation modes in cochlear implants, there are just two physical electrodes-one known as the “active” electrode, and the other known as the “reference” electrode (also referred to as the “indifferent” electrode). Current flows between the two electrodes by whatever path is dictated by the impedances of the structures surrounding the electrodes. However, in principle, either the active electrode or the reference electrode could consist of more than one physical electrode shorted together. In the common ground stimulation mode, for example, the indifferent (or common ground) electrode consists of all the intracochlear electrodes, other than the active electrode, shorted together by the electronics inside the implant. Another example is where two electrodes become accidentally shorted. Effectively, the two shorted electrodes can be thought of as a single, physically separate electrode, so if current is programmed to flow to one or the other of the two electrodes, the current actually ends up flowing to both. In cases like this where more than one physically separate electrode is shorted to another electrode, current will take separate paths to each of the electrodes, which affects the recorded response.

The principal of superposition holds that each current path can be treated as separate, and the overall response will look like the sum of the responses from all the individual current paths. For example, assume that a short occurs between electrodes 1 and 7. In this scenario, approximately half the current flows from electrode 5 to 7, and half flows from electrode 5 to 1. The two current paths result in opposite polarity responses because one is in the basal to apical direction and the other is in the apical to basal direction. When the responses from the two paths are added together, the path from electrode 5 to 1 dominates because it is a wider mode and also more basal (closer to the cochleostomy). Therefore, the overall response is reversed in polarity (abnormal/unexpected response) compared to the response of the path where no evidence of malfunction existed (normal/expected response).

### Integrity Tests

The following section describes various integrity tests used by the integrity testing system for cochlear implants. For each test, an example of a waveform without evidence of malfunction is described, along with an explanation of why the waveform looks the way it does based on the primary effects or principles explained in the preceding section above.

(1) Common Ground Scan Test. The common ground scan test checks the function of each electrode in common ground mode.

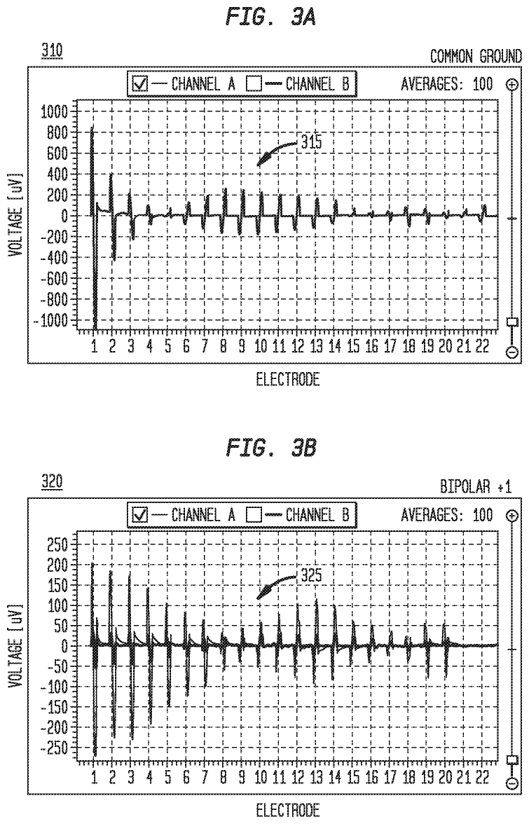

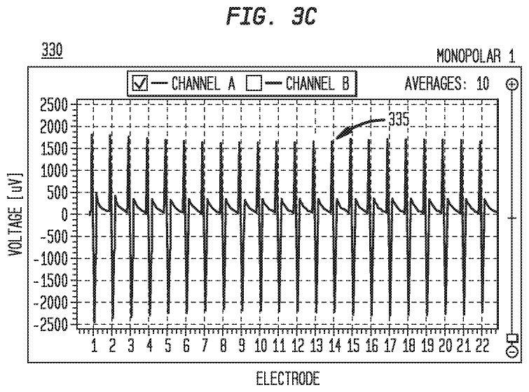

FIG. 3A is a common ground test scan plot 310 for an implant, showing a normal response 315 for a healthy cochlea and no evidence of malfunction. As shown in FIG. 3A, the normal expected result 315 is one biphasic waveform for each active electrode tested. Response amplitudes are large for the most apical electrodes in one polarity, then taper to zero amplitude or a “null” around electrodes 6-10, then increase in amplitude but in opposite polarity to the most apical electrodes. Typically, the normal response 315 should have large amplitude pulses in the basal region (left side, lower numbered electrodes), which smoothly decrease in amplitude to a null around electrode 6-10. From the null to the apical region (right side, higher numbered electrodes), the amplitude of the normal response 315 should smoothly increase again with the pulses having opposite polarity to those in the basal region.

Generally, when stimulated in common ground mode, the normal response 315 should appear to have an “hour glass” shape (i.e., large amplitudes at either end, with a null somewhere near the middle). It is noted that the null point where the current flowing in both directions tend to cancel each other out is normally more basal of the geometrical center electrode on the array, because the more basal current paths have a stronger influence on the overall response than the more apical current paths.

In contrast to the normal result 315 shown in FIG. 3A, an electrode array with short circuit(s) or open circuit(s) will typically appear in the common ground test as a pulse that has an atypical amplitude compared to surrounding electrodes (abnormal result), as described further below. Open circuits will often appear as very small spikes (not related to noise), rather than the expected biphasic waveform. Short circuits will usually appear as a very small phase one amplitude and an atypically large phase two amplitude.

Additionally, the common ground test can be analyzed with a bipolar+1 test and/or a monopolar test to help identify cochlear abnormalities (such as cochlear otospongiosis or a misplaced electrode array), examples of which are described further below. Note that with the common ground test, phase two often has a larger amplitude than phase one (even for a normally functioning device), particularly for more apical electrodes, and phase two can even be several times the size of phase one. This is not an indication of a problem with the device, and is only the case for certain devices in common ground mode (but not some other devices, and not in other modes such as bipolar or monopolar modes).

(2) Bipolar+1 Scan Test. The bipolar+1 scan test checks the function of each electrode in a bipolar+1 mode. The bipolar+1 scan test is mainly used in conjunction with the common ground scan test, as a cross-check for detecting electrode faults and identifying any “sponginess” or other cochlear pathologies.

FIG. 3B is a bipolar+1 test scan plot 320 for an implant, showing a normal response 325 for a healthy cochlea and no evidence of malfunction. As shown in FIG. 3B, the normal expected result 325 is one biphasic pulse pair for each electrode. Amplitudes should be large at the basal end (left side of plot) and diminish or reduce at the apical end (right side of plot). The polarity of the pulses should all be in the same direction.

In contrast to the normal result 325 shown in FIG. 3B, electrode shorts in the bipolar+1 test usually appear as atypical amplitudes compared with the surrounding electrodes, and electrode opens typically appear as the absence of pulses or as small spikes in place of the normal pulses (abnormal results), as described further below. Faults on a particular electrode will also appear on the electrode two positions more basal (e.g., if electrode 10 is faulty, then the short will show upon electrode 8 as well as electrode 10, because in bipolar+1 mode, electrode 8 uses electrode 10 as its reference/indifferent electrode).

The bipolar+1 plot is one of the most sensitive of the electrode tests for irregularities in the electrode array or the cochlea itself. Often amplitudes will vary from electrode to electrode caused by small differences in electrode separation, or perhaps by positioning or slight kinking of the electrode array, or variation of the structure within the cochlea. It is also noted that the bipolar+1 test uses the narrowest stimulation mode of all the electrode tests, and therefore reveals the most about how the electrode array or the cochlea itself changes along its length. The origin of the reducing amplitude from basal to apical electrodes comes directly from the fact that the amplitude of the response diminishes with distance from the cochleostomy, as explained above.

(3) Monopolar (Monopolar 1 & Monopolar 2) Scan Test. The monopolar 1 scan test checks the function of each electrode using the extracochlear electrode 1 (or ECE 1) as the reference/indifferent electrode. Note that the ECE 1 is also known as the monopolar 1 (or MP1) electrode. The monopolar 1 scan test is primarily used to check the integrity of the monopolar 1 electrode and to provide information on the extent of any cochlear otospongiosis, malformation, or any abnormality in the placement of the electrode array (e.g., a partially extruded array).

FIG. 3C is a monopolar 1 test scan plot 330 for an implant, showing a normal response 335 for a healthy cochlea and no evidence of malfunction. As shown in FIG. 3C, the normal expected result 335 is one biphasic waveform for each active electrode tested. All responses should have roughly the same amplitude for each electrode. In a healthy cochlea where there is correct electrode array placement, most of the current passes from the ECE 1 (MP1) to the intracochlear electrode via the cochleostomy. Hence, the amplitude of the response 335 is not significantly affected by the electrode number, since the direction of current is from the ECE 1 (MP1) to the cochleostomy regardless of the electrode number. In cases where there is no evidence of malfunction, the response 335 has similar amplitudes across the entire electrode array, as shown in FIG. 3C.

In contrast to the normal result 335 shown in FIG. 3C, an abnormal unexpected result for the monopolar 1 test is where the response amplitude varies for each electrode. In cases where there is evidence of a malfunction, the response amplitude varies across the electrode array, sometimes reversing in phase (an abnormal phase reversal). Further examples of this are described below.

In the monopolar 1 test, current is passed from each intracochlear electrode to and from the ECE 1 (MP1) extracochlear electrode. Because the current passes outside the cochlea and hence near to the recording electrode(s), the signal is large compared to recordings made using other modes with only intracochlear electrodes (e.g., common ground mode and bipolar+1 mode), so a lower starting gain setting is used.

The monopolar 2 test is similar to the monopolar 1 test, but uses the extracochlear electrode 2 (or ECE 2) as the reference/indifferent electrode. The ECE 2 is also known as the monopolar 2 (or MP2) electrode or the plate electrode. Note that it is normal for the monopolar 2 test to have a different amplitude (and/or occasionally, a different polarity) to that of the monopolar 1 test. This is because of the different locations of the ECE 1 (MP1) and ECE 2 (MP2) electrodes and the different current paths that occur as a consequence. Often in the case of cochlear abnormalities (e.g., sponginess) or incorrect positioning of the electrode array, the monopolar 1 and 2 plots can look quite different. Nevertheless, the key point with both monopolar tests to look for is whether or not the response amplitudes are relatively constant across the electrode array.

### Finding Evidence of Malfunction (Faults)

In the next section, several faults that have been found in cochlear implants are described (including but not limited to electrode shorts to stiffening rings, shorts between electrodes, an extruded array, and a spongy cochlea), along with explanation regarding how these electrode faults or other abnormalities are manifested in the results of an integrity test. This section is organized by fault type, rather than by each integrity test, because many faults manifest themselves as atypical measurements that can be better understood and interpreted across multiple different tests. Hence, a particular fault can be characterized and verified as it will tend to appear as a series of atypical measurements in a number of different tests. In one aspect, confidently predicting a malfunction with a cochlear implant involves forming a hypothesis about a suspected fault from one test result, and then cross-referencing that hypothesis with other test results where that fault is likely to appear.

In the common ground and bipolar+1 tests, the test results should be analyzed to look for smooth transitions in amplitude across the entire electrode array. Any amplitudes that vary dramatically from a smoothly changing pattern can have suspect electrodes that are potentially malfunctioning. Although the overall shape of the scan plots can vary dramatically due to cochlear pathologies and variations in electrode array placement, these variations typically cause “smooth” changes in amplitude, whereas abrupt changes in amplitude are usually indicative of electrode faults.

A test which best shows any atypical measurement is selected, and a hypothesis of the malfunction is formed using the common fault models described herein. That malfunction can then be cross-checked with other tests and/or recipient information (e.g., programming, use, etiology, medical and surgical information, imaging, performance results, longitudinal electrode impedance measurements, etc.) that would support the hypothesis. For each test that confirms the fault hypothesis, a confidence level associated with the hypothesis increases. Conversely, a confidence level associated with the hypothesis can decrease for each test that conflicts with (contradicts or does not confirm) the fault hypothesis. In either case, the information that either supports or does not support the hypothesis can be recorded and stored for use in future analysis.

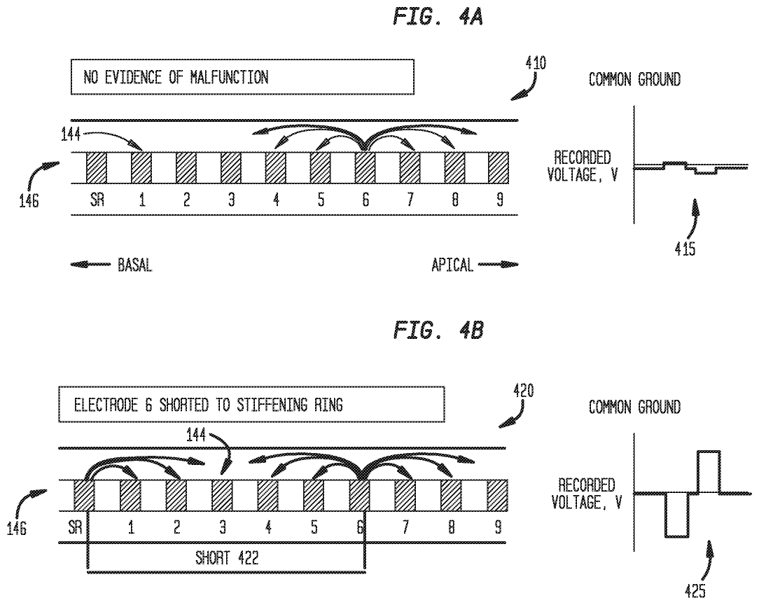

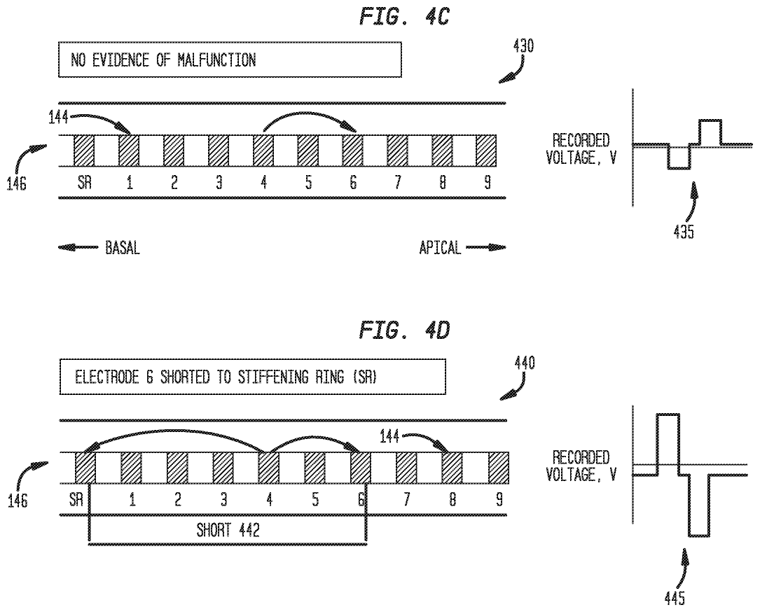

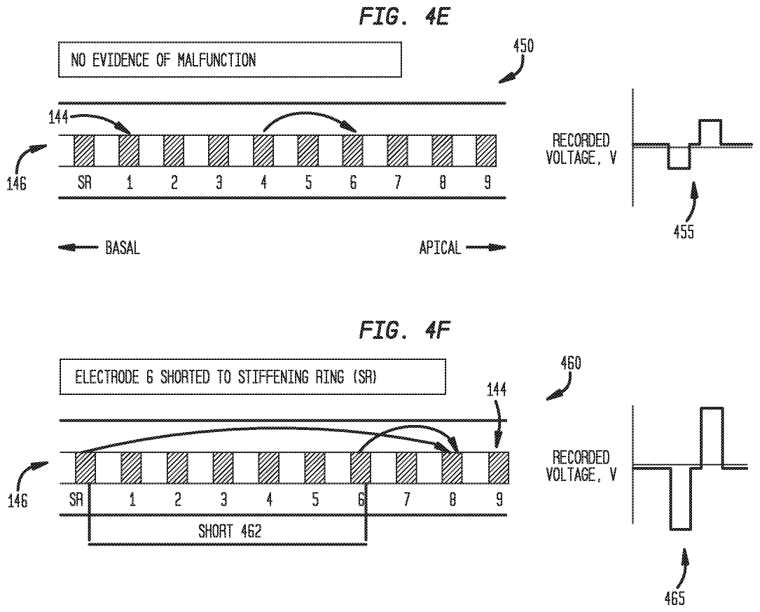

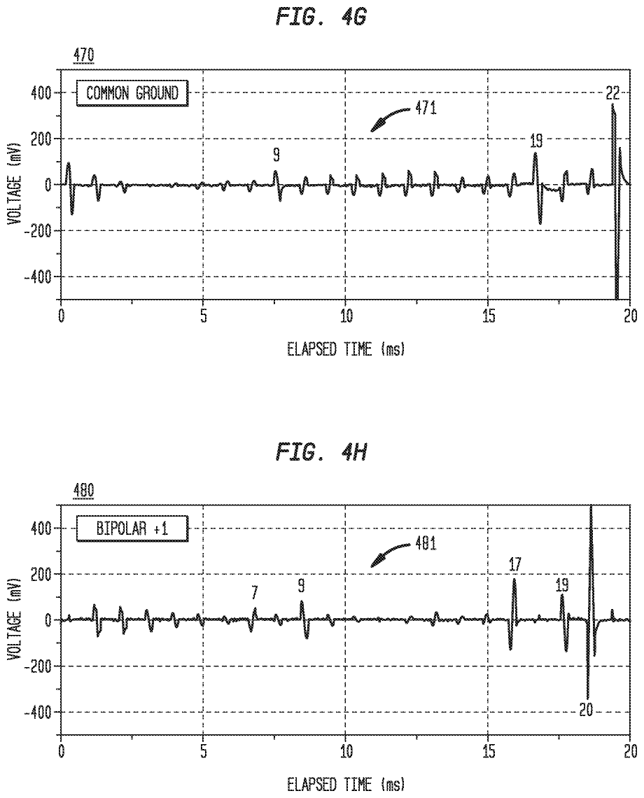

(1) Electrode shorts to stiffening rings. Shorts of electrodes to stiffening rings can occur from time to time, particularly in older devices. Various examples of electrode shorts to stiffening rings are described below with reference to FIGS. 4A-4H.

Common ground test. A short to a stiffening ring in common ground mode normally results in a large pulse of the same polarity as pulses at the most basal (low numbered, left hand side) end of the common ground test scan plot. In a properly set up integrity test system, this is a negative phase one pulse.

Normal result. FIG. 4A is a diagram showing normal current flow 410 and the recorded voltage response 415 when there is no evidence of malfunction in common ground mode. FIG. 4A shows the normal situation where current flows from a given stimulating electrode (e.g., electrode 6) to all the other electrodes of the array, the recorded response is small because the current flows in both directions and therefore the apical to basal current flow and basal to apical current flow tend to cancel each other out. In this example, although there are more electrodes in the apical direction than the basal direction from the given electrode (e.g., electrode 6) such that a little more of the current would flow in the basal to apical direction, the basal electrodes are closer to the cochleostomy (less deep into the cochlea) so they tend to have a larger effect on the response. The result is an approximate cancellation or nulling of the recorded response resulting a very small response amplitude.

Abnormal result. FIG. 4B is a diagram showing abnormal current flow 420 and the effect on the recorded voltage response 425 when there is a short 422 from electrode 6 to a stiffening ring in common ground mode. As shown in FIG. 4B, in an abnormal situation where the given stimulating electrode (e.g., electrode 6) is shorted to a stiffening ring (e.g., the most apical stiffening ring in this example), the current flows from the stiffening ring as well as the given electrode (e.g., electrode 6) to all of the other electrodes in the array. Because the stiffening ring is more basal than all the other electrodes of the array, all the current flow from the stiffening ring is in the basal to apical direction. The stiffening ring is also less far away from the cochleostomy than the other electrodes, so the stiffening ring contributes more significantly to the overall response. The net superposition of all the currents is to produce a large response in the polarity that represents basal to apical current flow. Usually this stands out very clearly from the neighboring pulses in the common ground test scan plot.

Bipolar+1 test. A short to a stiffening ring in bipolar+1 mode causes a large pulse of opposite polarity to the pulse on the electrode two or more basal than the faulty electrode (i.e., the electrode that uses the faulty electrode as a reference electrode in bipolar+1 mode), and also causes an atypically large pulse of the same polarity as the normal pulse on the faulty electrode.

Normal result. FIG. 4C is a diagram showing normal current flow 430 and the recorded voltage response 435 when there is no evidence of malfunction when stimulating on electrode 4 in bipolar+1 mode. In the bipolar+1 mode, a similar situation to the common ground mode occurs. In bipolar+1 mode, every electrode in the test for a normal device shows a pulse of the same polarity since the current is basal to apical (in phase two). As shown in FIG. 4C, in the normal situation, current flows basal to apical producing a relatively small response (modest pulse corresponding to a basal to apical current flow direction), because bipolar+1 is not a particularly wide mode.

Abnormal result. In an abnormal situation where the given stimulating electrode (e.g., electrode 6) is shorted to a stiffening ring, the response from electrode 4 as well as electrode 6 is affected, since a bipolar+1 mode pulse on electrode 4 uses electrode 6 as its indifferent electrode. Therefore, a characteristic of all faults in BP+1 mode plots (not just limited to shorts to stiffening rings) is that an atypical amplitude is seen on the faulty electrode as well as the electrode two more basal than the faulty electrode. Note that the exact magnitude of the effects will vary depending on exactly which stiffening ring and which electrode are shorted.

FIG. 4D is a diagram showing abnormal current flow 440 and the effect on the recorded voltage response 445 when there is a short 442 from electrode 6 to a stiffening ring when stimulating on electrode 4 in bipolar+1 mode. As shown in FIG. 4D, if electrode 6 is shorted to a stiffening ring while stimulating on electrode 4, some of the current flows from electrode 4 to the stiffening ring (the exact amount of the current depends on the relative impedances of the two paths) flows from electrode 4 to the stiffening ring. This produces a pulse of the opposite polarity since the current flow for this path is apical to basal. This also has a much larger contribution to the overall response, because the mode is wider and the path is closer to the cochleostomy. This path dominates the overall response and the result is a large pulse of opposite polarity (compared to the normal case with no evidence of malfunction).

FIG. 4E is a diagram showing normal current flow 450 and the recorded voltage response 455 when there is no evidence of malfunction when stimulating on electrode 6 in bipolar+1 mode. As shown in FIG. 4E, the normal result in the case of no evidence of malfunction produces a modest pulse corresponding to a basal to apical current flow direction. FIG. 4F is a diagram showing abnormal current flow 460 and the effect on the recorded voltage response 465 when there is a short 462 from electrode 6 to a stiffening ring when stimulating on electrode 6 in bipolar+1 mode. As shown in FIG. 4F, if electrode 6 is shorted to a stiffening ring while stimulating on electrode 6, a second path is created, but in the same basal to apical direction in this instance. Again, the path is wide and close to the cochleostomy, so it dominates the overall response, but in this instance its polarity is the same as that of the normal case with no evidence of malfunction. Its effect is therefore to dramatically increase the size of the response, but to leave its polarity unchanged.

FIG. 4G is a common ground test scan plot 470 for an implant, showing an abnormal response 471 when there is a short circuit from one or more stimulating electrodes (electrodes 9, 19, and 22 in this example) to a stiffening ring in common ground mode. FIG. 4H is a bipolar+1 test scan plot 480 for an implant, showing an abnormal response when 481 there is a short circuit from one or more stimulating electrodes (electrodes 9, 19, and 22 in this example) to a stiffening ring in bipolar+1 mode. As described above, anomalous pulse amplitudes and polarities can be indicative of the existence of shorts to stiffening rings (e.g., at electrodes 9, 19, and 22 in FIGS. 4G and 4H). Note that there can be some variation in the response amplitudes of other (non-faulty) electrodes across a given test scan plot. There seems to be a particularly abrupt change in amplitude between neighboring electrodes 15 and 16, for example. However, cross-checking with other test scan plots reveals no particular pattern that fits any of the common fault models described above. In this instance, this variation is smoother than the abrupt changes of electrodes 9, 19, and 22, and is probably caused by spongy (i.e., porous) cochlear bone, as described further below. This highlights a difficult problem in electrode fault identification-how to tell which electrodes have genuinely atypical amplitudes caused by faults and those which are just part of the natural variation in amplitude caused by irregularities in the cochlear structures.

Thus, in the case of electrode fault determination, it is desirable to cross-check a hypothesis from one test scan plot with one or more other test scan plots that would be expected to show anomalies due to the hypothesized fault (e.g., analyze the common ground test scan plot and the bipolar+1 test scan plot and interpret them both together). In some instances, it can be desirable to further cross-check recorded impedances (e.g., longitudinal electrode impedance data captured using a clinical electrode testing tool) for any suspect electrode that is potentially malfunctioning, as well as any medical imaging that can be available (e.g., x-ray, CT, MRI).

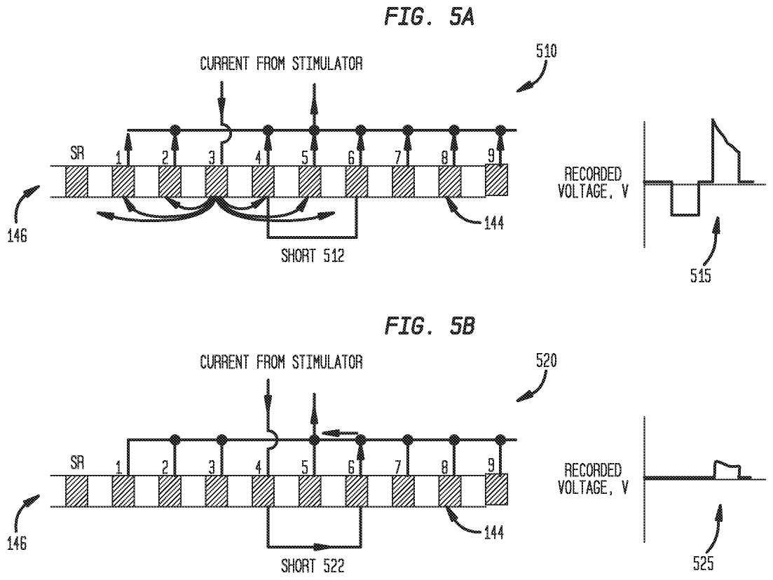

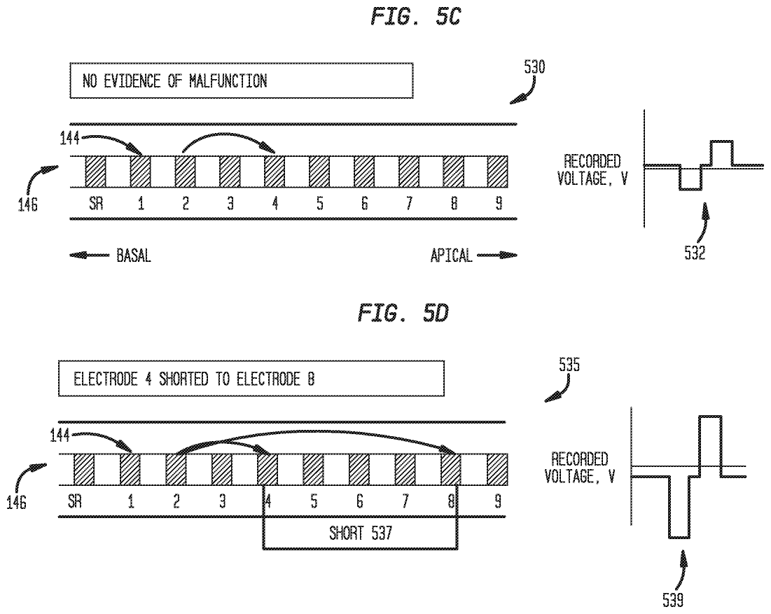

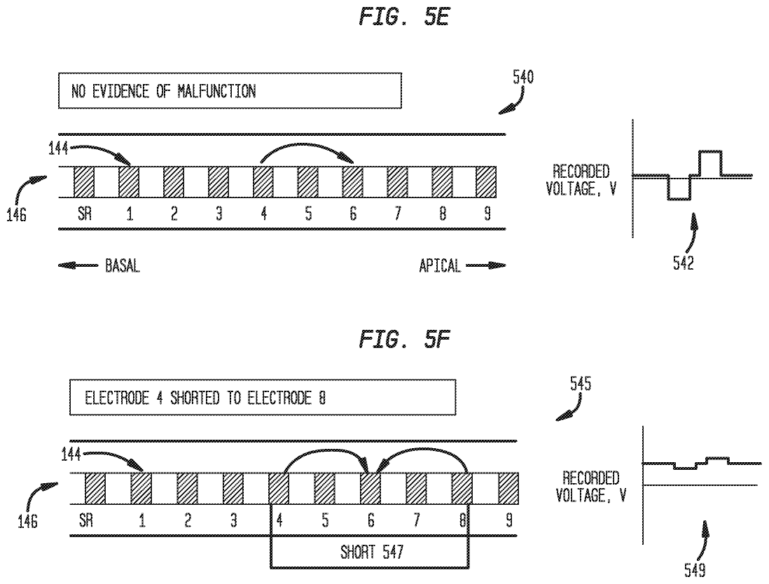

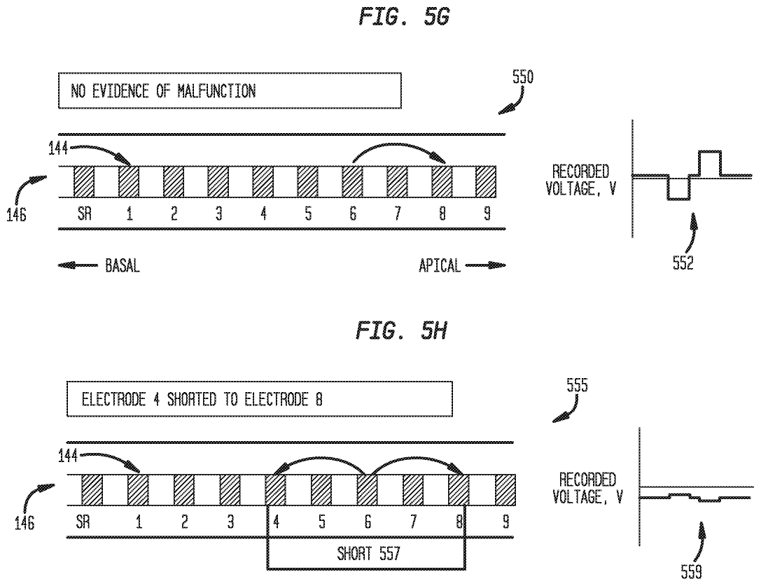

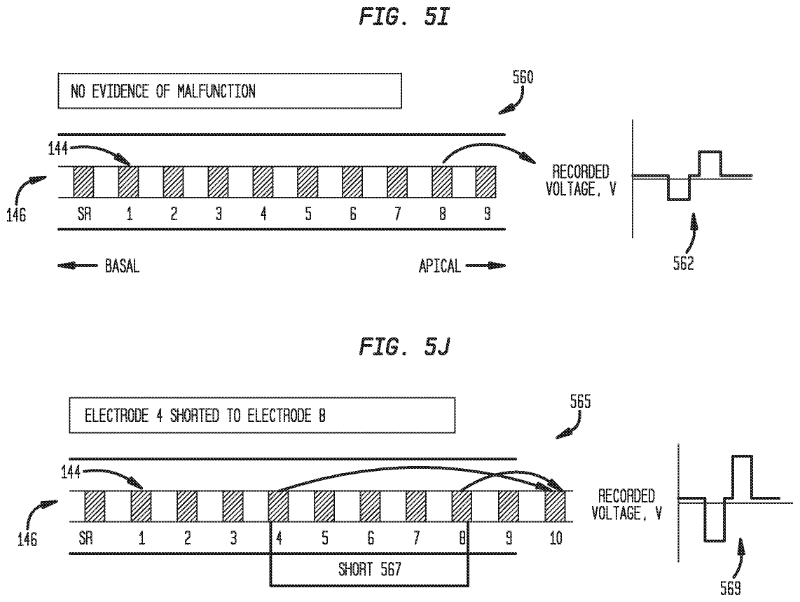

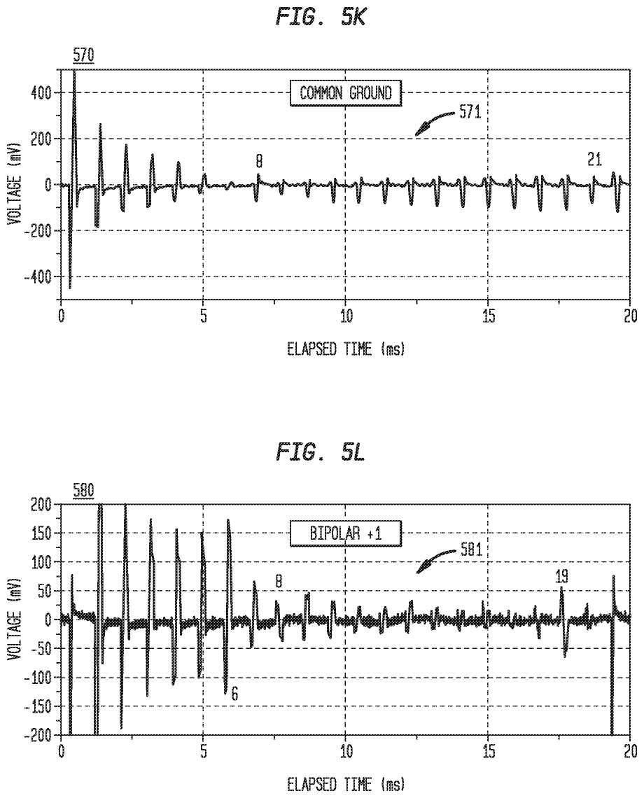

(2) Shorts between electrodes. In the case of shorts between electrodes, the short provides an alternate current path (or paths) to the expected path, and the overall response can be calculated as a superposition of the responses from all the individual current paths. In principle, any electrode can be shorted to any other electrode, such that there are more variations of faults and the way each of these faults manifests themselves for shorts between electrodes than there are for shorts to stiffening rings. Again, this highlights the importance of cross-checking a given fault model against the different tests, and possible other available data, to confirm validity of the fault hypothesis. Various examples of shorts between electrodes are described below with reference to FIGS. 5A-5L.

Electrode shorts to other electrodes in common ground mode behave differently from electrode shorts to stiffening rings, and this different behavior can be useful in distinguishing shorts between electrodes and shorts to stiffening rings. In common ground mode, a short to another electrode produces no response (or a small response with very little amplitude) in phase one of the pulse. Phase two is often modified and can or cannot be present, and phase two can even increase in amplitude due to the short in some cases. However, the phase one behavior is fairly reliable and can often be used to identify electrode shorts to other electrodes.

Electrode shorts to other electrodes in common ground mode. A shorted electrode in the common ground test scan appears as having an absent (or small) phase one response. The phase two response for a shorted electrode can be smaller, the same, or larger than the normal response expected for a non-faulty electrode.

FIG. 5A is a diagram showing current flow 510 and how a short 512 between electrodes in common ground mode causes current to flow through tissue (rather than the short 512) when stimulating on a non-shorted electrode (electrode 3 in this example). As shown in FIG. 5A, when stimulating on a non-shorted electrode (e.g., electrode 3), current flows from the active electrode through tissue to reach the common ground electrode. In this case, shorts between other electrodes in the array do not affect the current flow since all the other electrodes are grounded together anyway.

FIG. 5B is a diagram showing current flow 520 and how a short 522 between electrodes in common ground mode causes current to flow through the short 522 (rather than tissue) when stimulating on the shorted electrode (electrode 4 in this example). As shown in FIG. 5B, when a shorted electrode is used as the active electrode (e.g., electrode 4), a short exists to one of the other electrodes (e.g., electrode 6). Since this other electrode is part of the common ground electrode, a short now exists directly between the active and common ground electrodes, and current flows through this shorting path rather than through tissue. Since no current flows through tissue, no response is observed. In reality, however, shorts are not necessarily zero impedance, so some small response can be observed, and phase two will also often be present.

When there is no evidence of electrode malfunction, the common ground test has a natural null around electrodes 6 to 10 (refer to FIG. 3A), which can make it difficult to detect atypical measurements in that region because the pulse amplitudes are so small there. In this case, the bipolar+1 test can be used to help resolve any doubts if electrode faults are suspected in this region.

Electrode shorts to other electrodes in bipolar+1 mode. Shorts between electrodes occur on at least two electrodes, so any shorts are usually expected to produce at least four atypical pulse amplitudes on electrodes in the bipolar+1 test plot. A fault on the more basal of a pair of shorted electrodes will cause a pair of atypical pulses, two electrodes apart. The lower numbered pulse (two electrodes lower than the shorted electrode) will be larger in amplitude than expected, and the higher numbered pulse (the shorted electrode) will be smaller in amplitude or phase reversed. A fault on the more apical of a pair of shorted electrodes will cause a pair of atypical pulses, two electrodes apart. The lower numbered pulse (two electrodes lower than the shorted electrode) will be smaller in amplitude than expected or phase reversed, and the higher numbered pulse (the shorted electrode) will be larger in amplitude.

The bipolar+1 scan test provides a useful crosscheck of shorts that are suspected from the common ground scan test, and it is particularly useful if only a small number of faults exist. When multiple shorts are present, or where a few shorts occur close together on the array, it can be difficult to interpret the bipolar+1 test because the number of atypical measurements on electrodes can easily equal or exceed the number of non-faulty electrodes in one region of the array. Each fault affects two electrodes so it only takes two or three shorts in one region to make it difficult to pick the atypical measurements from the background “normal” trend.

FIGS. 5C-5J show the effects on the pulse amplitude of a short that occurs between electrodes 4 and 8. Stimulation in bipolar+1 mode is shown for electrode 2 (FIGS. 5C and 5D), electrode 4 (FIGS. 5E and 5F), electrode 6 (FIGS. 5G and 5H), and electrode 8 (FIGS. 5I and 5J), since they are the four electrodes affected by this fault in this non-limiting illustrative example.

Normal result. FIG. 5C is a diagram showing normal current flow 530 and the recorded voltage response 532 when there is no evidence of malfunction when stimulating on electrode 2 in bipolar+1 mode. FIG. 5E is a diagram showing normal current flow 540 and the recorded voltage response 542 when there is no evidence of malfunction when stimulating on electrode 4 in bipolar+1 mode. FIG. 5G is a diagram showing normal current flow 550 and the recorded voltage response 552 when there is no evidence of malfunction when stimulating on electrode 6 in bipolar+1 mode. FIG. 5I is a diagram showing normal current flow 560 and the recorded voltage response 562 when there is no evidence of malfunction when stimulating on electrode 8 in bipolar+1 mode. As shown in FIGS. 5C, 5E, 5G, and 5I, the normal result in the case of a healthy cochlea and no evidence of malfunction is a moderately small response (because BP+1 is a relatively narrow mode) of negative polarity.

Abnormal result. The abnormal result depends on which electrode is being stimulated, as explained below. The basic principles of wider current paths producing larger responses and of superposition can be used to predict the atypical responses when an electrode is shorted to another electrode.

FIG. 5D is a diagram showing abnormal current flow 535 and the effect on the recorded voltage response 539 when there is a short 537 from electrode 4 to electrode 8 when stimulating on electrode 2 in bipolar+1 mode. As shown in FIG. 5D, the abnormal result when stimulating on electrode 2 is that the short causes another current path of wider mode (e.g., bipolar+5) because the indifferent electrode is shorted to a more apical electrode. Hence, the overall response increases in amplitude. In this instance, it roughly doubles because the superposition of the BP+1 path and the BP+5 path gives an amplitude approximating a BP+3 path. This is about twice the width of the BP+1 path in the case of a normal result with no evidence of malfunction. The exact extent of the increase depends on the location of the two shorted electrodes (e.g., the wider apart they are, the larger the increase will be). However, the more basal path will also tend to dominate, so both effects should be considered to predict the final response.

FIG. 5F is a diagram showing abnormal current flow 545 and the effect on the recorded voltage response 549 when there is a short 547 from electrode 4 to electrode 8 when stimulating on electrode 4 in bipolar+1 mode. As shown in FIG. 5F, the abnormal result when stimulating on electrode 4 is that the more apical electrode now causes an apical to basal current from electrode 8 to electrode 6. This works to reduce the current flowing in the normal path from electrode 4 to electrode 6. In this instance, as the paths are the same widths and in the opposite direction, they roughly cancel each other out. In general, a short to a more apical electrode will tend to reduce the response amplitude or even reverse its phase when stimulation is applied to the affected electrode.

FIG. 5H is a diagram showing abnormal current flow 555 and the effect on the recorded voltage response 559 when there is a short 557 from electrode 4 to electrode 8 when stimulating on electrode 6 in bipolar+1 mode. As shown in FIG. 5H, the abnormal result when stimulating on electrode 6 is a decrease or phase reversed amplitude compared to the normal result of FIG. 5G when the electrode is not shorted. FIG. 5J is a diagram showing abnormal current flow 565 and the effect on the recorded voltage response 569 when there is a short 567 from electrode 4 to electrode 8 when stimulating on electrode 8 in bipolar+1 mode. As shown in FIG. 5J, the abnormal result when stimulating on electrode 8 is an increased amplitude compared to the normal result of FIG. 5I when the electrode is not shorted. In the cases of FIGS. 5H and 5J, because the short is to an electrode more basal than electrode 8, the resulting changes in amplitude are reversed.

FIG. 5K is a common ground test scan plot 570 for an implant, showing an abnormal response 571 when there is a short circuit between two electrodes in common ground mode. FIG. 5L is a bipolar+1 test scan plot 580 for an implant, showing an abnormal response 581 when there is a short circuit between two electrodes in bipolar+1 mode. In these non-limiting illustrative examples, the implant has a short from electrode 8 to electrode 21.

As shown in the common ground plot 570 of FIG. 5K, electrodes 8 and 21 appear to break from the background trend somewhat (based on a comparison with their neighboring electrodes). Closer inspection reveals that their phase-ones are also zero, although in the case of electrode 8 this can be due to the fact that the natural null in the plot occurs here (not necessarily due to a short). Phase two for electrode 8 is unusually large. For electrode 21, phase one is zero, which is clearly atypical when compared to adjacent electrodes, and phase two is smaller than adjacent electrodes. These characteristics of the common ground plot 570 of FIG. 5K are indicative of a short between electrodes (hypothesis), and another plot (e.g., bipolar+1) can be analyzed as well for confirmation of the hypothesis that there is a short between electrodes 8 and 21.

As shown in the bipolar+1 plot 580 of FIG. 5L, which gives a clearer indication of the existence of a short between electrodes, electrode 6 is larger than normal and electrode 8 is phase reversed. This is consistent with the pattern expected for the most basal of a pair of shorted electrodes. Likewise, electrode 19 is phase reversed, which is consistent with electrode 21 being the most apical of a pair of shorted electrodes. Note that there is no bipolar+1 plot for electrode 21, because this would require stimulation using electrode 23 (which does not exist). Therefore, all the evidence from the BP+1 plot of FIG. 5L confirms the hypothesis from analyzing the common ground plot of FIG. 5K that there is, in fact, a short between electrodes 8 and 21.

In addition, impedance measurement functionality (i.e., being able to review a longitudinal set of impedance measurement data) and/or electrode testing functionality can also provide evidence of open and short circuit electrodes, and can also be reviewed in conjunction with the common ground and bipolar+1 integrity tests to assist with interpreting electrode information for a given case.

(3) Extruded electrodes. One or more integrity test scans (possibly in combination with impedance measurement data) can also provide a useful guide as to whether the electrode array is partially extruded or fully outside the cochlea.

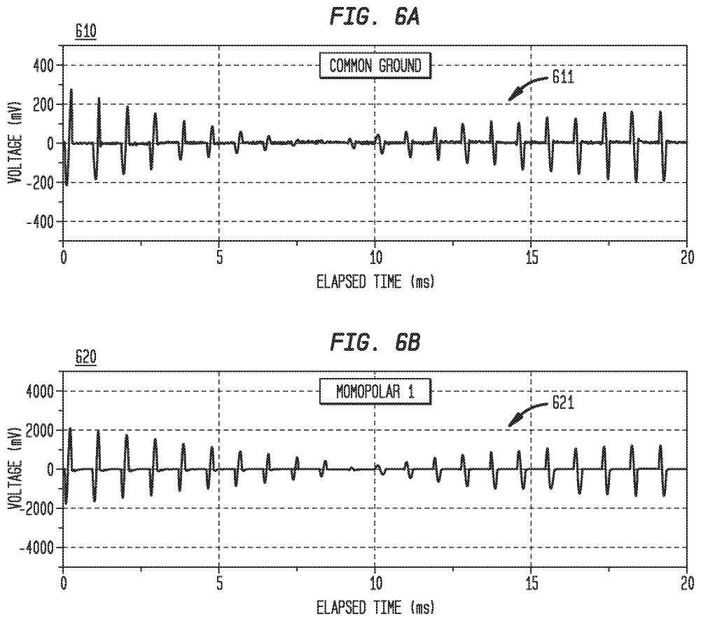

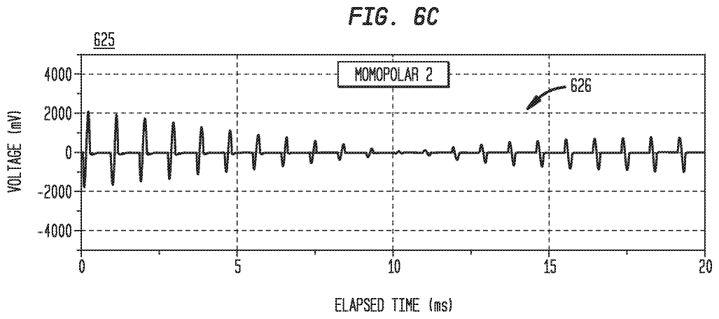

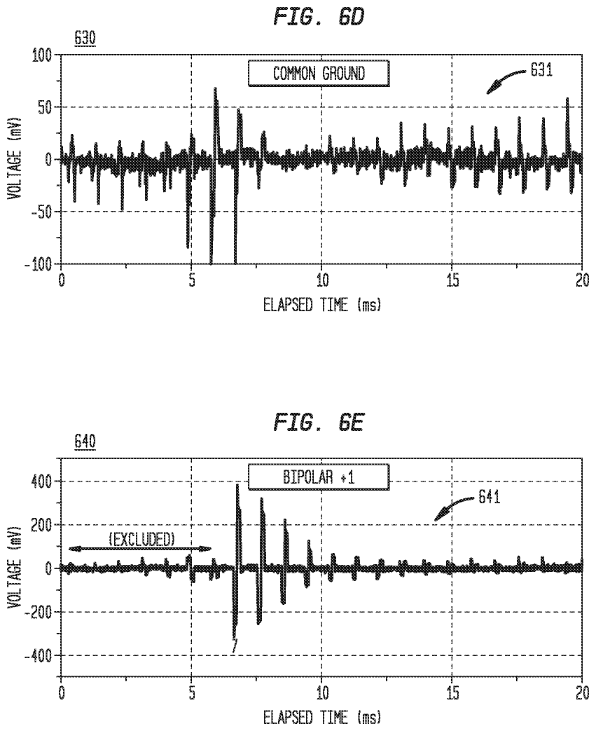

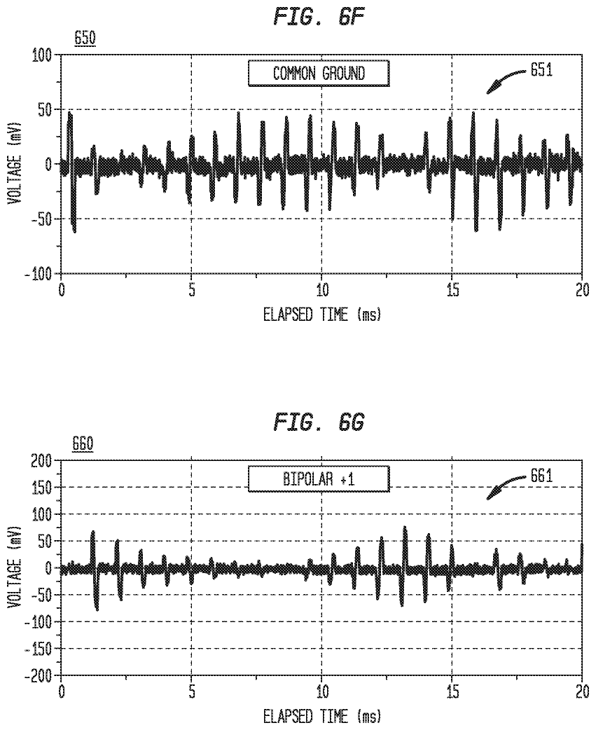

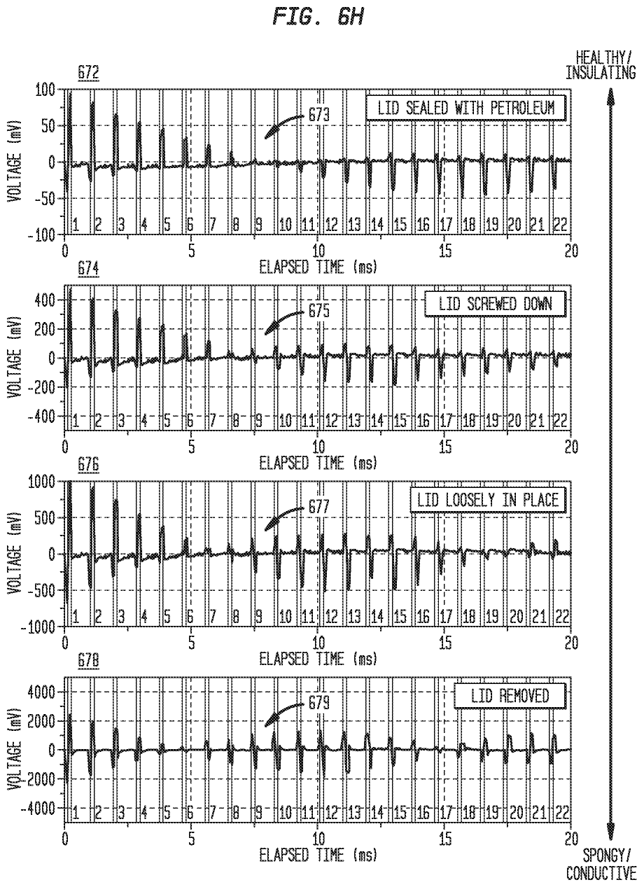

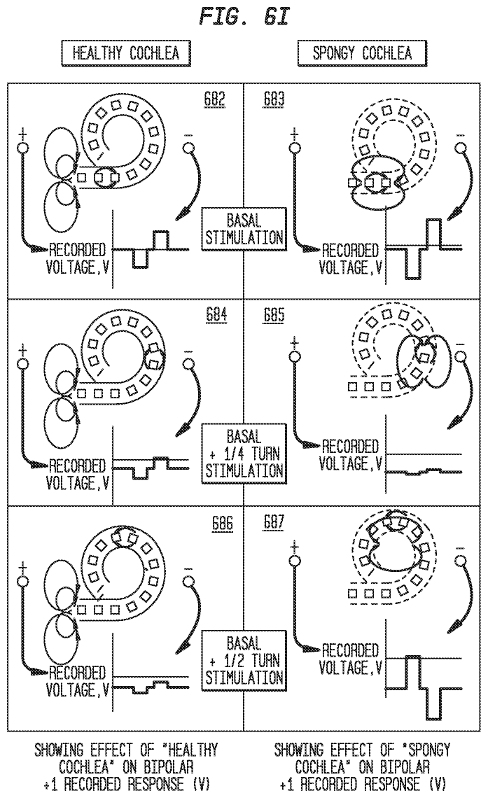

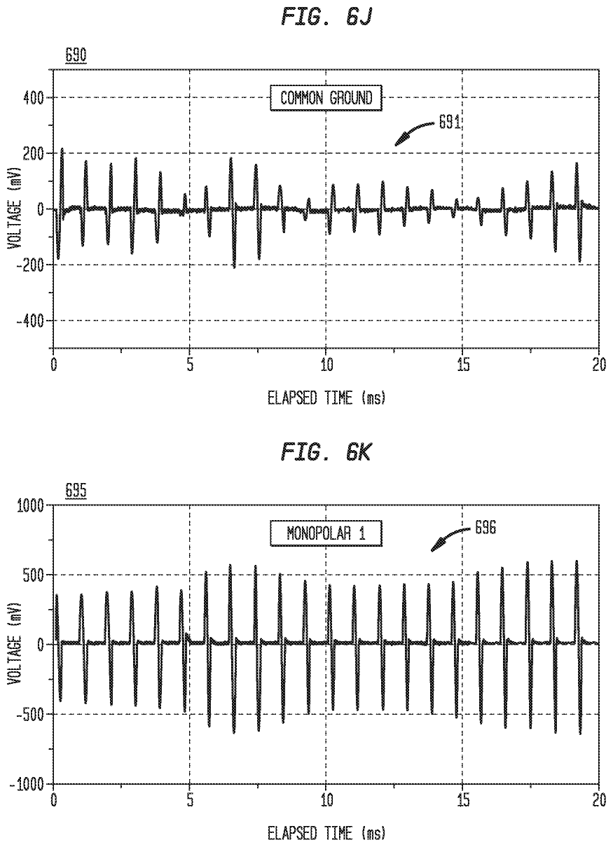

Common ground test. FIG. 6A is a common ground test scan plot 610 for an implant, showing an abnormal response 611 when the electrode array is completely outside the cochlea. In the case of an electrode array that is completely outside the cochlea, the common ground test scan plot can show normal (or near normal) results.

Monopolar test. FIGS. 6B and 6C are monopolar 1 and monopolar 2 test scan plots 620 and 625 for an implant, showing abnormal responses 621 and 626, respectively, when the electrode array is completely outside the cochlea. The monopolar tests (MP1 or MP2) shown in FIGS. 6B and 6C are both far from normal in the case of a partially extruded electrode array. A normal monopolar 1 plot (refer to FIG. 3C) or a normal monopolar 2 plot should have pulse amplitudes and phases that are all about the same or substantially similar. However, in the abnormal monopolar 1 plot 620 of FIG. 6B and the abnormal Monopolar 2 plot 625 of FIG. 6C, the amplitudes vary dramatically across the array and the pulses change phase at the null point near the middle of the array. This is a sure sign of either an extruded array, or else a very spongy cochlea. In either case, current flows directly to the intracochlear electrode, and as the electrode position changes through the scan, so does the direction of current flow and the evoked response. In a healthy cochlea (not spongy) and a full insertion of the electrode array (not extruded), current flows to the cochlea opening (e.g., cochleostomy), irrespective of the intracochlear electrode being stimulated, as described above.

However, in some cases it can be difficult to determine solely from an integrity test similar to the tests shown in FIGS. 6A, 6B and 6C whether the cochlea is spongy, or the array is extruded. Generally, an x-ray and the clinical symptoms can be used to distinguish a case of an extruded electrode array from a case of a spongy cochlea more conclusively. If the array is extruded, there will often be no auditory sensation, even at high stimulus levels. In the case of a spongy cochlea, hearing will typically occur at normal or sometimes low stimulus levels, but it will sometimes be accompanied by side effects (e.g., facial nerve stimulation or pain).

FIG. 6D is a common ground test scan plot 630 for an implant, showing an abnormal response 631 for the case of an implant recipient known to have a partially extruded electrode array. FIG. 6E is a bipolar+1 test scan plot 640 for an implant, showing an abnormal response 641 when an electrode array is partially extruded from the cochlea. In this case, it appears that the array exits the cochlea at around electrode 7. The common ground plot 630 of FIG. 6D is much noisier than the bipolar+1 plot 640 of FIG. 6E because it was recorded at a current level of 1. One of the symptoms for this particular recipient was pain on the basal electrodes at very low current levels, so this was the loudest current that could be used. The bipolar+1 plot 640 of FIG. 6E was taken at higher current levels but with electrodes 1 through 6 “excluded,” so that output on electrodes 1 to 6 is restricted to current level 1. In the common ground plot 630 of FIG. 6D, very little response is seen on electrodes 1 to 5, which is probably because these electrodes are outside the cochlea and cannot be in good contact with tissue (effectively, they are open circuit). Around electrode 7, the more normal common ground response can be seen to start with a null around electrode 10. The bipolar+1 plot 640 of FIG. 6E also shows a rapid decrease in amplitude from electrode 7, again indicative of electrode 7 being near the cochleostomy.

(4) Spongy cochlea. Certain diseases of the cochlea, such as otosclerosis, are known to be associated with a condition known as cochlear otospongiosis (sometimes referred to as “spongy” bone or a spongy cochlea). When the bone is spongy, it is less dense and more porous than normal, and is therefore more conductive. For example, the results of a common ground test and a bipolar+1 test can be analyzed to determine if the test scan plots show characteristics consistent with porous bone (i.e., evidence of a spongy cochlea).

FIG. 6F is a common ground test scan plot 650 for an implant, showing an abnormal response 651 for the case of an implant recipient known to have otosclerosis (resulting in cochlear otospongiosis, or spongy cochlea bone) in common ground mode. FIG. 6G is a bipolar+1 test scan plot 660 for an implant, showing an abnormal response 661 in the case of cochlear otospongiosis (spongy cochlea) in bipolar+1 mode. This recipient was complaining of “jolts” and other non-auditory sensations at the time the test was made. In particular, there are two phase inversions that occur in the common ground and bipolar+1 plots of FIGS. 6F and 6G, and these phase inversions are indicative of a spongy cochlea. It is also noted that, allowing for a little random variation from electrode to electrode, the amplitudes change relatively smoothly, progressing from one phase to the other then back again. There are no abrupt changes in amplitude to suggest that electrode faults can account for the odd overall shape of the waveform.

The reason for the unusual overall shape of the common ground plot 650 of FIG. 6F and the bipolar+1 plot 660 of FIG. 6G is because the spongy nature of the cochlea bone in these cases makes it behave more like a conductor, rather than an insulator. When this happens, the idealized theories which explain the normal curves described above no longer apply, and the electrode array behaves more as if it were in an open volume of conducting fluid, rather than in a conducting tube.

FIG. 6H shows a series of four common ground test scan plots 672, 674, 676, and 678, taken using an electrode array inserted into a model cochlea immersed in saline, to simulate different responses 673, 675, 677, and 679 ranging from a healthy insulating cochlea to a spongy conductive cochlea. This example simulates making the model cochlea progressively less insulating and more conductive from the top plot down to the bottom plot. The model had a removable lid that could be screwed down with different degrees of tightness, simulating an electrically conductive cochlea when the lid was loose (so a conducting path of liquid existed from the cochlear duct to the external fluid) and an insulating cochlea when the lid was screwed down tight. The top plot shows the situation with the lid screwed down tight, simulating a healthy, insulating cochlea. The bottom plot is with the lid removed entirely (but with the electrode array still curled in the cochlea duct of the model), which effectively simulates a very conductive (spongy) cochlea. The overall shape of the plots changes from something approaching that of the normal common ground plot (refer to FIG. 3A) to a plot which looks similar to the abnormal common ground plot from the otosclerotic cochlea (refer to FIG. 6F), as the cochlea goes from being electrically insulating in the top plot of FIG. 6H to electrically conductive in the bottom plot of FIG. 6H.

In some example embodiments where a computer-based automated diagnostic analysis process occurs, simulations like the one described with reference to FIG. 6H and others can be used to train one or more models (e.g., AI, ML, NN, etc.) with normal training data sets and abnormal training data sets to recognize and differentiate between the various faults and abnormalities described herein.

The above-described phenomenon occurs in all the test scan plots, but it is easiest to understand with reference to bipolar+1 mode. FIG. 6I shows a series of panels 682-687 illustrating current flow and the effect on the recorded voltage response in phase two of the stimulation pulse for a normal healthy cochlea (left hand side) and a spongy cochlea (right hand side) when stimulating at three different positions along the electrode array in bipolar+1 mode. The top two panels show the situation for basal stimulation, the middle two panels depict stimulation about one quarter turn more apical, and the bottom two panels show the case for stimulation half a turn more apical than the top panels.

In the normal healthy cochlea (left hand side of FIG. 6I), the bony walls of the cochlea constrain the current flow, and the recording electrodes pick up a voltage induced by current flow outside the cochleostomy. As stimulation is advanced more basally, current flow from the cochleostomy reduces, and the amplitude of the recorded response reduces proportionately, as described above. For the spongy cochlea (right hand side of FIG. 6I) the cochlear walls do not constrain the current, so the current flow follows a direction dictated by the stimulating electrode pair. As the electrode position advances more basally, the direction of the electrode pair rotates with the curve of the cochlea, and the recorded response varies accordingly. Stimulation at opposite sides of the cochlear duct (compare the top and bottom panels) therefore results in a recorded response of opposite polarity. It also follows that the stimulation must go through a null at some point in order for the polarity to reverse (depicted in the middle panel in FIG. 6I).

FIG. 6J is a common ground test scan plot 690 for an implant, showing a suspect response 691 in the case of a suspected spongy cochlea in common ground mode. In the case of an implant recipient with a suspected spongy cochlea, with the common ground plot shown in FIG. 6J, the situation is complicated somewhat by the multiple current paths that flow, but a similar situation still holds and double or even triple nulls have been observed. FIG. 6K is a monopolar 1 test scan plot 695 for an implant, showing a suspect response 696 in the case of a suspected spongy cochlea in monopolar 1 mode. In the Monopolar 1 and 2 plots shown in FIG. 6K, the oscillations are usually less pronounced because the monopolar electrode is some distance from the cochlea, which reduces the change in current direction still further. Nevertheless, the characteristic steady undulation can still be observed in some monopolar plots (such as the one shown in FIG. 6K, for example). In this case, the common ground plot of FIG. 6J has three nulls, whereas the monopolar 1 plot of FIG. 6K shows only relatively small undulations.

In some cases, the hypothesis regarding the presence of spongy cochlea bone can be confirmed with a preoperative CT scan, for example, which should show evidence of a decrease in bone density in the otic capsule.

### Example Variations and Alternatives

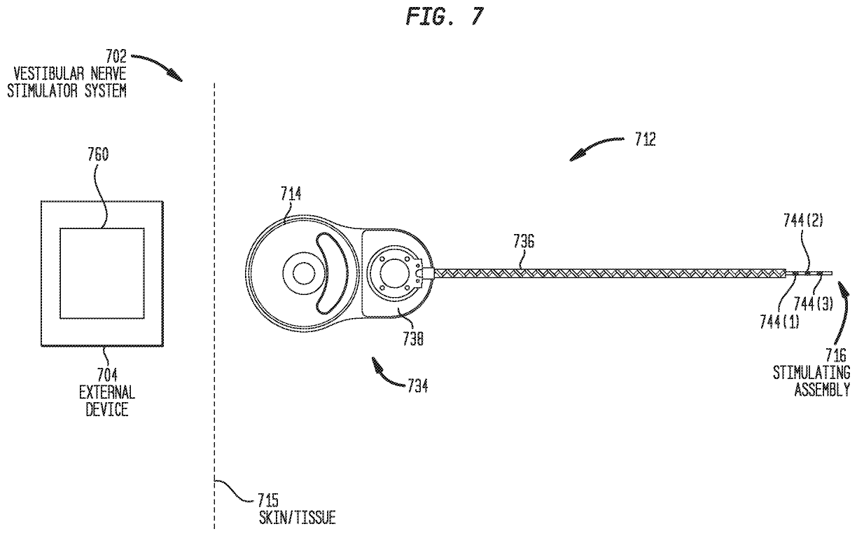

As previously described, the technology disclosed herein can be applied in any of a variety of circumstances and with a variety of different devices. Example devices that can benefit from technology disclosed herein are described in more detail in FIGS. 7 and 8 below. As described below, the operating parameters for the devices described with reference to FIGS. 7 and 8 can be configured according to the techniques described herein. For example, the techniques described herein can be used to prioritize clinician tasks associated with configuring the operating parameters of wearable medical devices, such as a vestibular stimulator as described in FIG. 7, or an implantable stimulation system as described in FIG. 8. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue.

FIG. 7 illustrates an example vestibular nerve stimulator system 702, with which embodiments presented herein can be implemented. As shown, the vestibular nerve stimulator system 702 comprises an implantable component (vestibular stimulator) 712 and an external device/component 704 (e.g., external processing device, battery charger, remote control, etc.). The external device 704 comprises a transceiver unit 760. As such, the external device 704 is configured to transfer data (and potentially power) to the vestibular stimulator 712.

The vestibular stimulator 712 comprises an implant body (main module) 734, a lead region 736, and a stimulating assembly 716, all configured to be implanted under the skin (tissue) 715 of the recipient. The implant body 734 generally comprises a hermetically-sealed housing 738 in which RF interface circuitry, one or more rechargeable batteries, one or more processors, and a stimulator unit are disposed. The implant body 134 also includes an internal/implantable coil 714 that is generally external to the housing 738, but which is connected to the transceiver via a hermetic feedthrough (not shown).

The stimulating assembly 716 comprises a plurality of electrodes 744(1)-(3) disposed in a carrier member (e.g., a flexible silicone body). In this specific example, the stimulating assembly 716 comprises three (3) stimulation electrodes, referred to as stimulation electrodes 744(1), 744(2), and 744(3). The stimulation electrodes 744(1), 744(2), and 744(3) function as an electrical interface for delivery of electrical stimulation signals to the recipient's vestibular system.

The stimulating assembly 716 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 712, the external device 704, and/or another external device, can be configured to implement the techniques presented herein. That is, the vestibular stimulator 712, possibly in combination with the external device 704 and/or another external device, can include an evoked biological response analysis system, as described elsewhere herein.

FIG. 8 is a functional block diagram of an implantable stimulator system 800 that can benefit from the technologies described herein. The implantable stimulator system 800 includes the wearable device 100 acting as an external processor device and an implantable device 30 acting as an implanted stimulator device. In examples, the implantable device 30 is an implantable stimulator device configured to be implanted beneath a recipient's tissue (e.g., skin). In examples, the implantable device 30 includes a biocompatible implantable housing 802. Here, the wearable device 100 is configured to transcutaneously couple with the implantable device 30 via a wireless connection to provide additional functionality to the implantable device 30.

In the illustrated example, the wearable device 100 includes one or more sensors 812, a processor 814, a transceiver 818, and a power source 848. The one or more sensors 812 can be one or more units configured to produce data based on sensed activities. In an example where the stimulation system 800 is an auditory prosthesis system, the one or more sensors 812 include sound input sensors, such as a microphone, an electrical input for an FM hearing system, other components for receiving sound input, or combinations thereof. The processor 814 can be a component (e.g., a central processing unit) configured to control stimulation provided by the implantable device 30. The stimulation can be controlled based on data from the sensor 812, a stimulation schedule, or other data. Where the stimulation system 800 is an auditory prosthesis, the processor 814 can be configured to convert sound signals received from the sensor(s) 812 (e.g., acting as a sound input unit) into signals 851. The transceiver 818 is configured to send the signals 851 in the form of power signals, data signals, combinations thereof (e.g., by interleaving the signals), or other signals. The transceiver 818 can also be configured to receive power or data. Stimulation signals can be generated by the processor 814 and transmitted, using the transceiver 818, to the implantable device 30 for use in providing stimulation.

In the illustrated example, the implantable device 30 includes a transceiver 818, a power source 848, and a medical instrument 811 that includes an electronics module 810 and a stimulator assembly 830. The implantable device 30 further includes a hermetically sealed, biocompatible implantable housing 802 enclosing one or more of the components.

The electronics module 810 can include one or more other components to provide medical device functionality. In many examples, the electronics module 810 includes one or more components for receiving a signal and converting the signal into the stimulation signal 815. The electronics module 810 can further include a stimulator unit. The electronics module 810 can generate or control delivery of the stimulation signals 815 to the stimulator assembly 830. In examples, the electronics module 810 includes one or more processors (e.g., central processing units or microcontrollers) coupled to memory components (e.g., flash memory) storing instructions that when executed cause performance of an operation. In examples, the electronics module 810 generates and monitors parameters associated with generating and delivering the stimulus (e.g., output voltage, output current, or line impedance). In examples, the electronics module 810 generates a telemetry signal (e.g., a data signal) that includes telemetry data. The electronics module 810 can send the telemetry signal to the wearable device 100 or store the telemetry signal in memory for later use or retrieval.

The stimulator assembly 830 can be a component configured to provide stimulation to target tissue. In the illustrated example, the stimulator assembly 830 is an electrode assembly that includes an array of electrode contacts disposed on a lead. The lead can be disposed proximate tissue to be stimulated. Where the system 800 is a cochlear implant system, the stimulator assembly 830 can be inserted into the recipient's cochlea. The stimulator assembly 830 can be configured to deliver stimulation signals 815 (e.g., electrical stimulation signals) generated by the electronics module 810 to the cochlea to cause the recipient to experience a hearing percept. In other examples, the stimulator assembly 830 is a vibratory actuator disposed inside or outside of a housing of the implantable device 30 and configured to generate vibrations. The vibratory actuator receives the stimulation signals 815 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 818 can be components configured to transcutaneously receive and/or transmit a signal 851 (e.g., a power signal and/or a data signal). The transceiver 818 can be a collection of one or more components that form part of a transcutaneous energy or data transfer system to transfer the signal 851 between the wearable device 100 and the implantable device 30. Various types of signal transfer, such as electromagnetic, capacitive, and inductive transfer, can be used to usably receive or transmit the signal 851. The transceiver 818 can include or be electrically connected to a coil 20.

As illustrated, the wearable device 100 includes a coil 108 for transcutaneous transfer of signals with the concave coil 20. As noted above, the transcutaneous transfer of signals between coil 108 and the coil 20 can include the transfer of power and/or data from the coil 108 to the coil 20 and/or the transfer of data from coil 20 to the coil 108. The power source 848 can be one or more components configured to provide operational power to other components. The power source 848 can be or include one or more rechargeable batteries. Power for the batteries can be received from a source and stored in the battery. The power can then be distributed to the other components as needed for operation.

As should be appreciated, while particular components are described in conjunction with FIG. 8, technology disclosed herein can be applied in any of a variety of circumstances. The above discussion is not meant to suggest that the disclosed techniques are only suitable for implementation within systems akin to that illustrated in and described with respect to FIG. 8. In general, additional configurations can be used to practice the methods and systems herein and/or some aspects described can be excluded without departing from the methods and systems disclosed herein.

### Analyzing and Interpreting Integrity Test Results

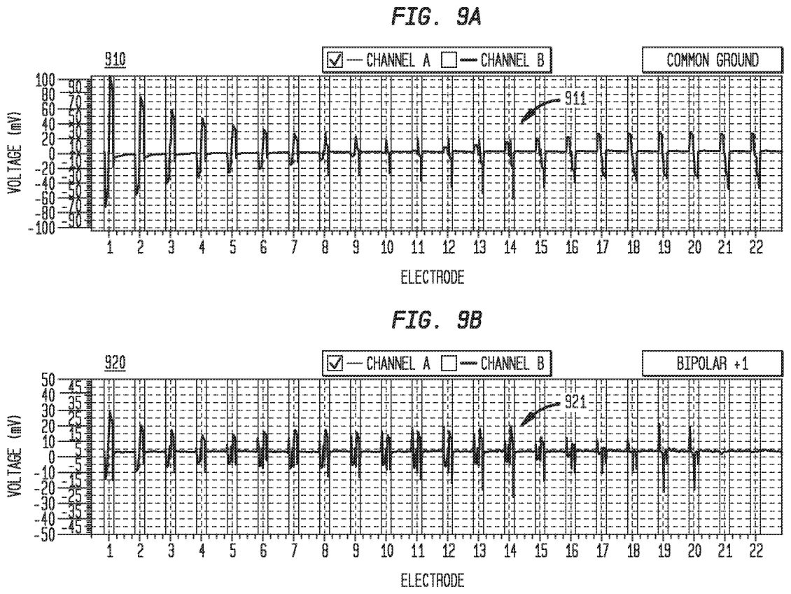

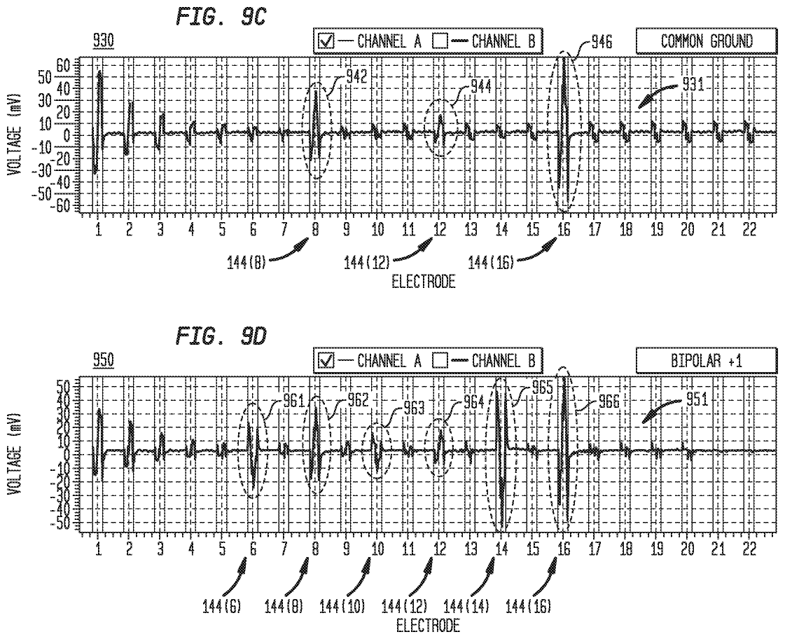

Next, a specific example describing how to interpret a set of integrity test plots is described with reference to FIGS. 9A-9D. The example of FIGS. 9A-9D illustrates what phase reversals can indicate, and also how to read and interpret the common ground and bipolar+1 test scan plots of the integrity testing system. As described elsewhere herein, the analysis and interpretation of these test scans can be a manual implementation (e.g., by an experienced medical professional), partially automated (e.g., a combination of human and computer hardware/software, or even fully automated (e.g., via the training of artificial intelligence models, machine learning algorithms, neural networks, or the like) according to different example embodiments.

FIG. 9A is a common ground test scan plot 910 for an implant, showing a normal voltage response 911 in the case of a healthy cochlea with no evidence of malfunction in common ground mode. FIG. 9B is a bipolar+1 test scan plot 920 for an implant, showing a normal voltage response 921 in the case of a healthy cochlea with no evidence of malfunction in bipolar+1 mode. In these examples, there are no electrode faults (undamaged electrode array). FIGS. 9A and 9B show the electric field as captured by one or more recording electrodes of the integrity testing system that results from the stimulation which leaks out of the electrode array entrance into the cochlea. The cochlea is not a perfect insulator, and there is the opening where the electrode enters. Because of this leakage, this electric field can be seen. The waveforms tend to be in line with the electric field leaking out of a single opening (the entrance) to an insulated cylindrical cavity.

The common ground plot 910 of FIG. 9A has an “hourglass” shape. The artefacts at the basal end are of the highest amplitude because the active electrode is both closest to the entrance to the cochlea, and all the current is flowing in one direction only (when considering one phase only). Moving along the array, some of the current is reversing in direction, as the active electrode is no longer at the end and the grounding electrodes are on both sides of it. Thus, the amplitude drops moving further along the array. Eventually there will be an equal amount of current flowing both basally and apically, which is where the common ground plot 910 of FIG. 9A has a null due to superposition of the currents flowing in opposition to each other. Note that the null does not occur at the exact halfway point along the array, because electrodes nearer to the cochlear entrance carry more “weight” in terms of the leakage of the electric field out of the cochlea being measured. Moving even more apically along the array, there is now more current flowing back and so now the phase of the artefacts has reversed (this is normal/expected here). Towards the apical end of the array, the amplitudes grow again as the superposition of currents becomes dominated by current flowing in the opposite direction.

The bipolar+1 plot 920 of FIG. 9B is not the same as the common ground plot 910 of FIG. 9A, in that each artefact is due to a single pair of electrodes, so the current should never be changing directions (referring to one phase only). The artefacts just become smaller in amplitude progressive deeper into the cochlea, until often they are undetectable (since there is minimal leakage of the electric field out of the cochlear entrance). Note, however, that if current is leaking out in other places, a change in phase when progressing deeper into the cochlea for a bipolar+1 test scan can represent the electrode wrapping around beyond 360 degrees insertion.

FIG. 9C is a common ground test scan plot 930 for an implant, showing an abnormal voltage response 931, and illustrating how to interpret the abnormal voltage response 931, in common ground mode. FIG. 9D is a bipolar+1 test scan plot 950 for an implant, showing an abnormal voltage response 951, and illustrating how to interpret the abnormal voltage response 951, in bipolar+1 mode. In these examples, there is a potential malfunction or fault with the electrode array. More specifically, phase reversals can be seen on isolated electrodes in FIGS. 9C and 9D, which can be indicative of short-circuit fault caused by a damaged electrode array (or possibly a spongy cochlea), for example.

The common ground plot 930 of FIG. 9C shows a pattern of decreasing amplitudes towards a null, which occurs around electrode 7 or electrode 8 (which would be a normal place for the null to occur), but there is actually a very large response 942 on electrode 8, which is also referred to as a phase reversal 942 herein. At this point, a normal expected amplitude on electrode 8 should be close to zero (or perhaps slightly positive). However, there is actually a sizeable negative amplitude (abnormal amplitude)—and hence, an abnormal phase reversal 942 at electrode 8. Moving farther along the electrode array in FIG. 9C, electrodes 9-11 appear normal (i.e., starting to grow in the positive direction, as expected). But then there is another phase reversal 944 at electrode 12 (not appearing as dramatic as electrode 8, but still similar in size considering where electrode 12's positive amplitude would have been expected). Then electrodes 13-15 appear normal, until reaching electrode 16 where there is another bigger phase reversal 946. Finally, electrodes 17-22 look normal.

When interpreted, the common ground plot 930 of FIG. 9C supports a hypothesis that electrodes 8, 12, and 16 have current flowing in the opposite direction to what is normal/expected during the common ground test scan, which means that when electrodes 8, 12, and 16 are the active electrodes being stimulated, there must be other electrodes involved too. The likely cause of this is a current shunt between each of these electrodes and another electrode (or some other current shunt to the implant case perhaps, or a stiffening ring if the model of array has stiffening rings).

The bipolar+1 plot 950 of FIG. 9D corroborates the hypothesis formed from analyzing and interpreting the common ground plot of FIG. 9C. In the bipolar+1 plot 950 of FIG. 9D, there are phase reversals 961, 963, and 965 on electrodes 6, 10 and 14, respectively, and abnormally larger amplitudes 962, 964, 966 in the “normal” direction on electrodes 8, 12 and 16. Recall that bipolar+1 mode means that electrode 6 stimulates to electrode 8, etc. This indicates that when stimulating on electrode 6 to electrode 8, current is flowing in the opposite direction from what was expected in the normal case. This also indicates that when stimulating on electrode 8 to electrode 10, more current is flowing in the “normal” direction than what was expected in the normal case.

When interpreted together, the bipolar+1 plot 950 of FIG. 9D further confirms the hypothesis that electrode 8 has a current shunt to something shallower in the cochlea (or even outside of the cochlea). The same conclusions can be made for electrode 12 and electrode 16. Thus, valuable diagnostic information can be provided via the detection and interpretation of phase reversals in the common ground and bipolar+1 tests considered together, for example.

It is also noted that phase reversals can and do occur due to strange anatomies, although they would likely appear as part of a pattern along the array, in contrast to the isolated phase reversals seen in the example of FIGS. 9C and 9D.

In some example embodiments, the techniques described above can involve some degree of manual interaction, such as by a medical professional having experience with viewing and interpreting the various test scan results that are produced by the system and displayed on a display screen of an external computing device, as described above. In some other example embodiments, the techniques described above can be at least partially automated, such as using artificial intelligence (AI) technology, machine learning (ML) algorithms, neural networks, or the like. The techniques involving AI/ML can be use a Big Dataset (large sample corpus of training data, test results, simulations, confirmed analyses, and the like are collected and stored over time) in order to create rules and train various AI/ML models to analyze and interpret the various integrity test scan results to identify, classify, and confirm various potential faults associated with electrode arrays of implants. These models can be updated and fine-tuned over time as further training data, test results, simulations, and/or confirmed analyses are obtained by the system.

In addition, obtaining as much background information as possible can also supplement interpretation of the results of integrity tests. For example, suspected otosclerosis or symptoms of facial nerve stimulation can account for an odd undulation in a waveform, or the fact that a recipient has Mondini's syndrome or a Common Cavity can explain a very unusual set of scan tests. In the manual case, knowledge of such background information can help the medical professional determine whether unexpected test results are due to faulty electrodes, a problem with the implant or telemetry, or rather some other anatomical or physiological issue, for example. In the automated case, such background information can be input into the AI/ML/NN models to make appropriate adjustments to various rules or parameters to account for the background information when analyzing, interpreting, and cross-checking integrity test scan results.

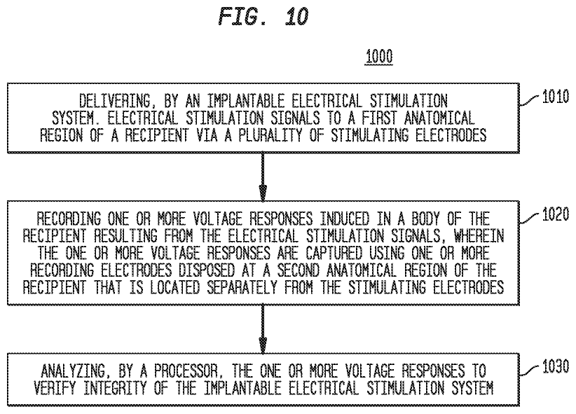

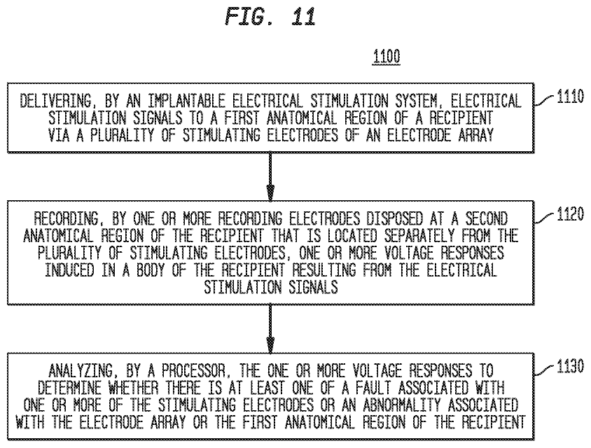

FIG. 10 is a flowchart illustrating a method 1000 for verifying integrity of an implantable electrical stimulation system, according to an example embodiment. Method 1000 begins at operation 1010, where the method includes delivering, by an implantable electrical stimulation system, electrical stimulation signals to a first anatomical region of a recipient via a plurality of stimulating electrodes.

At operation 1020, the method includes recording one or more voltage response induced in a body of the recipient resulting from the electrical stimulation signals, wherein the one or more voltage responses are captured using one or more recording electrodes disposed at a second anatomical region of the recipient that is located separately from the plurality of stimulating electrodes.

At operation 1030, the method further includes analyzing, by a processor, the one or more voltage responses to verify integrity of the implantable electrical stimulation system.

In some example embodiments, operation 1010 can include delivering a first set of electrical stimulation signals according to a first stimulus mode, and transmitting a second set of electrical stimulation signals according to a second stimulus mode, and operation 1020 can include recording a first set of voltage responses to the first set of electrical stimulation signals, and recording a second set of voltage responses to the second set of electrical stimulation signals. In this example, operation 1030 includes analyzing the first set of voltage responses in the first stimulus mode relative to the second set of voltage responses in the second stimulus mode to verify integrity of the implantable electrical stimulation system. In these example embodiments, the first stimulus mode is one of a common ground mode, a bipolar mode, or a monopolar mode, and the second stimulus mode is a different one of these modes. In one example, the first stimulus mode is a common ground mode and the second stimulus mode is a bipolar mode (e.g., bipolar+1). In another example, the first stimulus mode is a common ground mode and the second stimulus mode is a monopolar mode (e.g., monopolar 1 or monopolar 2).

In some examples, operation 1030 can include detecting an abnormal voltage response corresponding to one of the stimulating electrodes in relation to one or more voltage responses corresponding to one or more adjacent stimulating electrodes. In some examples, operation 1030 can include detecting an abnormal direction of current flow corresponding to one of the stimulating electrodes. In some examples, operation 1030 can include detecting an abnormal amplitude or an abrupt change in amplitude of one or more voltage responses corresponding to one or more of the stimulating electrodes. In some examples, operation 1030 can include detecting an abnormal phase reversal of one or more recorded voltage responses corresponding to one or more of the stimulating electrodes.

In some examples, operation 1030 can include identifying one or more electrode faults associated with one or more of the stimulating electrodes based on the one or more voltage responses. In one example, operation 1030 includes determining that there is a short circuit from one of the stimulating electrodes to a stiffening ring based on the one or more voltage responses. In another example, operation 1030 includes determining that there is a short circuit between two or more of the stimulating electrodes based on the one or more voltage responses.

In some examples, operation 1030 can include determining that at least a portion of an electrode array including the plurality of stimulating electrodes is extruded from the first anatomical region (e.g., the cochlea) of the recipient. In some examples, operation 1030 can include determining that the first anatomical region (e.g., the cochlea) of the recipient has an abnormal condition (e.g., cochlear otospongiosis, or “spongy” cochlea bone) based on the one or more voltage responses.

FIG. 11 is a flowchart illustrating a method 1100 for determining whether there is at least one of a fault associated with one or more stimulating electrodes or an abnormality associated with the electrode array or the first anatomical region of the recipient, according to an example embodiment.

At operation 1110, the method includes delivering, by an implantable electrical stimulation system, electrical stimulation signals to a first anatomical region of a recipient via a plurality of stimulating electrodes of an electrode array.

At operation 1120, the method includes recording, by one or more recording electrodes disposed at a second anatomical region of the recipient that is located separately from the plurality of stimulating electrodes, one or more voltage responses induced in a body of the recipient resulting from the electrical stimulation signals.

At operation 1130, the method further includes analyzing, by a processor, the one or more voltage responses to determine whether there is at least one of a fault associated with one or more of the stimulating electrodes or an abnormality associated with the electrode array or the first anatomical region of the recipient.

In some examples, the one or more recording electrodes are disposed at least a threshold separation distance from the plurality of stimulating electrodes. The threshold separation distance is a minimum distance between the stimulating electrodes and the one or more recording electrodes that permits phase reversals to be detected in the one or more voltage responses.

In some examples, operation 1130 can include determining that there is a short circuit fault associated with one or more of the stimulating electrodes in response to detecting an abnormal direction of current flow, an abnormal amplitude or an abrupt change in amplitude, an abnormal phase reversal, or a combination thereof based on the one or more voltage responses.

In some examples, operation 1130 can include determining that at least a portion of the electrode array including the plurality of stimulating electrodes is extruded from the first anatomical region (e.g., the cochlea) of the recipient in response to detecting an abnormal direction of current flow, an abnormal amplitude or an abrupt change in amplitude, an abnormal phase reversal, or a combination thereof based on the one or more voltage responses.

In some examples, operation 1130 can include determining that there is an abnormal condition of first anatomical region (e.g., the cochlea) of the recipient in response to detecting an abnormal direction of current flow, an abnormal amplitude or an abrupt change in amplitude, an abnormal phase reversal, or a combination thereof based on the one or more voltage responses.

Thus, the present disclosure has described an integrity testing system and corresponding methods for analyzing the functionality of an electrode array of a cochlear implant, identifying potential faults using one or more integrity test scans, classifying the potential faults as a particular category or type of fault, and cross-checking a hypothesis regarding the potential faults using one or more other integrity test scans. As described above, relative amplitudes (one pulse compared to another) as well as morphologies (shapes) of the responses are important features in diagnosing faults using the integrity test system and corresponding techniques described herein.

When a direction of current flow, an amplitude of the voltage, or a phase of the recorded voltage response is different than what is expected in the case of a normal healthy cochlea and an electrode array with no evidence of malfunction, these abnormal/unexpected test results (e.g., current flowing in the opposite direction, a substantially larger or smaller amplitude, or a complete 180 degree phase rotation compared to what was expected for the normal case) can be indicative of various electrode faults or anatomical problems. Whether the abnormal test results relate to abnormal direction of current flow, abnormal voltage amplitude, abnormal voltage phase reversals, or a combination thereof can be indicative of different categories or types of problems. In one example, the identification of an unexpected phase reversal where the direction of the current path is flipped around can indicate that the recipient has a “spongy cochlea” (rather than the implant having an electrode fault).

Additional integrity test scan results (e.g., a different test among common ground, bipolar, or monopolar) can also be used to cross-check and validate a specific hypothesis for explaining the abnormal test results. One particular benefit provided by the system and techniques described herein is removing ambiguity in interpretation of integrity test scan results, which is enabled at least in part by stimulating and measuring at one point in the recipient's body (e.g., current flow in the ear), and interpreting at another point (e.g., detecting voltage in the chest as the current flows out). Some example embodiments involve partial manual interpretation of test scan results (based on knowledge and experience), whereas some other example embodiments involve partial or fully automated interpretation of test scan results (based on artificial intelligence, machine learning, neural networks, and the like).

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method comprising:
delivering, by an implantable electrical stimulation system, electrical stimulation signals to a first anatomical region of a recipient via a plurality of stimulating electrodes;
recording one or more voltage responses induced in a body of the recipient resulting from the electrical stimulation signals, wherein the one or more voltage responses are captured using one or more recording electrodes disposed at a second anatomical region of the recipient that is located separately from the plurality of stimulating electrodes; and
analyzing, by a processor, the one or more voltage responses to verify integrity of the implantable electrical stimulation system.

2. The method of claim 1, wherein delivering the electrical stimulation signals to the first anatomical region of the recipient comprises:
delivering a first set of electrical stimulation signals according to a first stimulus mode; and
delivering a second set of electrical stimulation signals according to a second stimulus mode,
wherein recording the one or more voltage responses induced in the body of the recipient resulting from the electrical stimulation signals comprises:
recording a first set of voltage responses to the first set of electrical stimulation signals, and
recording a second set of voltage responses to the second set of electrical stimulation signals.

3. The method of claim 2, wherein analyzing the one or more voltage responses comprises:
analyzing the first set of voltage responses in the first stimulus mode in relation to the second set of voltage responses in the second stimulus mode to verify integrity of the implantable electrical stimulation system.

4. (canceled)

5. The method of claim 1, wherein analyzing the one or more voltage responses to verify integrity of the implantable electrical stimulation system comprises:
detecting an abnormal voltage response corresponding to one of the plurality of stimulating electrodes in relation to one or more voltage responses corresponding to one or more adjacent stimulating electrodes.

6. The method of claim 1, wherein analyzing the one or more voltage responses to verify integrity of the implantable electrical stimulation system comprises:
detecting an abnormal direction of current flow corresponding to one of the plurality of stimulating electrodes.

7. The method of claim 1, wherein analyzing the one or more voltage responses to verify integrity of the implantable electrical stimulation system comprises:
detecting an abnormal amplitude or an abrupt change in amplitude of one or more voltage responses corresponding to one or more of the plurality of stimulating electrodes.

8. The method of claim 1, wherein analyzing the one or more voltage responses to verify integrity of the implantable electrical stimulation system comprises:
detecting an abnormal phase reversal of one or more voltage responses corresponding to one or more of the plurality of stimulating electrodes.

9. The method of claim 1, wherein analyzing the one or more voltage responses to verify integrity of the implantable electrical stimulation system comprises:
identifying one or more electrode faults associated with one or more of the plurality of stimulating electrodes based on the one or more voltage responses.

10. (canceled)

11. (canceled)

12. The method of claim 1, wherein analyzing the one or more voltage responses to verify integrity of the implantable electrical stimulation system comprises:
determining that at least a portion of an electrode array including the plurality of stimulating electrodes is extruded from the first anatomical region of the recipient.

13. The method of claim 1, wherein analyzing the one or more voltage responses to verify integrity of the implantable electrical stimulation system comprises:
determining that the first anatomical region of the recipient has an abnormal condition based on the one or more voltage responses.

14. (canceled)

15. A system comprising:
an implantable electrical stimulation system configured to deliver electrical stimulation signals to a first anatomical region of a recipient via a plurality of electrodes that include at least one active electrode and at least one indifferent electrode;
one or more recording electrodes configured to record one or more voltage responses induced in a body of the recipient resulting from the electrical stimulation signals, wherein the one or more recording electrodes are disposed at a second anatomical region of the recipient that is not between the at least one active electrode and the at least one indifferent electrode; and
a processor configured to analyze the one or more voltage responses to verify integrity of the implantable electrical stimulation system.

16. The system of claim 15, wherein the one or more recording electrodes are implanted under a skin surface of the recipient.

17. The system of claim 16, wherein the one or more recording electrodes are integrated in a second implantable component including a battery, wherein the second implantable component is implanted under the skin surface of the recipient and is electrically connected to the implantable electrical stimulation system via a subcutaneous lead wire.

18. (canceled)

19. (canceled)

20. The system of claim 15, wherein the implantable electrical stimulation system is configured to deliver the electrical stimulation signals to the first anatomical region of the recipient via the plurality of electrodes in two or more different stimulation modes, and
wherein the processor is configured to analyze one or more voltage responses in each of the two or more different stimulation modes in relation to each other to verify integrity of the implantable electrical stimulation system.

21. (canceled)

22. The system of claim 20, wherein the processor is further configured to:
generate two or more integrity test scan plots for displaying the one or more voltage responses in each of the two or more different stimulation modes on a display of an external computing device.

23. The system of claim 15, wherein the processor is configured to analyze the one or more voltage responses to detect whether there is an abnormal voltage response corresponding to one of the plurality of electrodes in relation to one or more voltage responses corresponding to one or more adjacent plurality of electrodes to verify integrity of the implantable electrical stimulation system.

24. The system of claim 15, wherein the processor is configured to analyze the one or more voltage responses to detect whether there is an abnormal amplitude or an abrupt change in amplitude of one or more voltage responses corresponding to one or more of the plurality of electrodes to verify integrity of the implantable electrical stimulation system.

25. The system of claim 15, wherein the processor is configured to analyze the one or more voltage responses to detect whether there is an abnormal phase reversal of one or more voltage responses corresponding to one or more of the plurality of electrodes to verify integrity of the implantable electrical stimulation system.

26. The system of claim 15, wherein the processor is configured to identify one or more electrode faults based on one or more voltage responses corresponding to one or more of the plurality of electrodes.

27. (canceled)

28. The system of claim 15, wherein the processor is configured to determine that at least a portion of an electrode array including the plurality of electrodes is extruded from the first anatomical region of the recipient based on the one or more voltage responses.

29.-47. (canceled)
